(19) United States
(12) Patent Application Publication
(10) Pub. No.: US 2018/0136197 A1
(43) Pub. Date: May 17, 2018

(54) CELL-FREE METHODS OF DETECTING BIOACTIVE LIGANDS

(71) Applicant: Brigham Young University, Provo, UT (US)

(72) Inventors: Bradley C. Bundy, Provo, UT (US); Mark T. Smith, Logan, UT (US); David W. Wood, Columbus, OH (US); J. Porter Hunt, Provo, UT (US); Miriam J. Shakalli Tang, Provo, UT (US)

(21) Appl. No.: 15/813,026

(22) Filed: Nov. 14, 2017

Related U.S. Application Data
(60) Provisional application No. 62/421,622, filed on Nov. 14, 2016.
(60) Provisional application No. 62/526,721, filed on Jun. 29, 2017.

(51) Int. Cl. G01N 33/50; A61K 9/19

(52) U.S. Cl. CPC G01N 33/5023; C12N 15/70; C12Y 305/02006; A61K 9/19

(57) ABSTRACT

The present disclosure as disclosed in various embodiments is related to a bioassay for rapid screening and detection of bioactive ligands and screening for potential pharmaceuticals. In various embodiments are disclosed cell-free methods, systems, and kits for detecting bioactive ligands such as endocrine disrupting chemicals or nuclear hormone receptor modulators in a biological or environmental sample or for screening compounds for potential pharmaceutical application such as potential nuclear hormone receptor modulator pharmaceuticals.

### CROSS-REFERENCE TO RELATED APPLICATIONS

This application claims the benefit of U.S. Provisional Application Ser. No. 62/421,622 filed 14 Nov. 2016, and U.S. Provisional Application Ser. No. 62/526,721 filed 29 Jun. 2017, the disclosures of which are hereby incorporated in their entirety by reference herein.

### STATEMENT REGARDING FEDERALLY SPONSORED RESEARCH OR DEVELOPMENT

The invention was made with Government support under Grant Nos. 1R21ES16630; D13AP000037; and 1254148. The Government has certain rights to the invention.

### SEQUENCE LISTING

The text file Sequences_001_ST25.txt of size 46 KB created 14 Nov. 2017, filed herewith, is incorporated in its entirety by reference herein.

### TECHNICAL FIELD

The present disclosure as disclosed in various embodiments is related to a bioassay for rapid screening and detection of bioactive ligands and screening for potential pharmaceuticals. In various embodiments are disclosed cell-free methods, systems, and kits for detecting bioactive ligands such as endocrine disrupting chemicals or nuclear hormone receptor modulators in a biological or environmental sample or for screening compounds for potential pharmaceutical application such as potential nuclear hormone receptor modulator pharmaceuticals.

### BACKGROUND

Nuclear hormone receptors (NHRs) help regulate vital functions of the cells and organisms, such as metabolism, homeostasis, differentiation, development, and reproduction^{1,2,3}. NHRs interact with many natural and synthetic ligands and about 4% of all currently marketed therapeutics interfere with the activity of one or more NHRs⁴. NHRs also can interact with environmental endocrine disrupting chemicals (EDCs), which have become a public safety concern due to their ability to disrupt naturally occurring endocrine control. EDCs affect the endocrine system in humans and animals, commonly by mimicking natural hormones and binding to specific NHR ligand binding domains⁵.

Unintentional as well as intentional discharge of harmful chemicals into the environment has been the conventional reality of industrialized society for hundreds of years. In recent decades, an increasing wealth of evidence has shown EDCs to be of concern⁶. Studies have detected significant levels of EDC activity in air⁷, soil⁸, drinking water⁹, food¹⁰, personal care products¹¹, pharmaceuticals¹², and synthetic hormones¹². These studies suggest that EDC exposure likely contributes to acute and chronic conditions including cancer¹³, diabetes¹⁴, obesity^{11,15}, metabolic syndrome¹⁶, infertility¹¹, and permanent brain damage¹¹. A recent report estimated an EDC-exposure health burden of $340 billion USD in the United States and $209 billion USD in the EU¹⁴.

One class of EDC's known as xenoestrogens (XEs) interferes specifically with the function of estrogen receptors. XEs originate from both natural (e.g. soy plants) and unnatural (e.g. BPA) sources. Research has linked exposure to XEs with obesity¹⁷, birth defects¹⁸ including DNA methylation and placental alteration¹⁹, cancer²⁰, reproductive impairment²¹, cognitive disabilities^{22,23}, and developmental disorders²⁴. Thus, public chemical safety would be enhanced with rapid, reliable, and cost-effective methods to screen chemicals, environmental samples, and human/animal samples for high levels of XE activity.

Characterizing XE interactions with estrogen receptors also benefits medical technology. Pharmaceuticals called selective estrogen receptor modulators are currently used to treat a variety of conditions including infertility, breast cancer, and postmenopausal complications, and are one of the World Health Organization's “essential medicines”^{25,26}. Rapid screening technologies for ER modulators are valuable tools in drug discovery and characterization. Detection of estrogens and their derivatives in blood and urine samples is also an important diagnostic tool^{27,28}.

Long-standing methods for detecting XEs utilize yeast and human cell lines^{29,30,31,32,33,34,35}. While these are reliable and sensitive, their complicated laboratory procedures and long assay durations prohibit rapid screening and in-field detection^{12,36,37}. LC/MS and GC/MS are likewise popular techniques, but require trained technicians and significant equipment (˜$190,000)^{38,39,40}. Strategies employing biosensor proteins have been investigated in whole-cell⁴¹ and purified-protein formats⁴², but these methods require mammalian cell culturing and protein purification, both of which are cumbersome processes. There is a need for rapid and inexpensive methods for identifying XEs.

In order to deliver faster detection of NHR-ligand interactions, we previously developed an EDC biosensor platform where the presence of an EDC is reported through a change in growth phenotype of an engineered Escherichia coli strain^{43,44} or as disclosed in U.S. Pat. No. 7,592,144 is incorporated in its entirety by reference. This platform relies on a multi-domain engineered allosteric fusion protein, which reports ligand binding to a given NHR through the activation of a fused thymidylate synthase reporter enzyme. In practice, the biosensor protein is constitutively expressed in an engineered E. coli thymidine-auxotroph strain, leading the growth phenotype of the strain to be dependent on the presence of an NHR-targeting ligand. Binding of the ligand to the NHR ligand binding domain activates the thymidylate synthase reporter enzyme and enables cell growth, allowing the presence and activity of a specific NHR ligand to be readily ascertained by a simple turbidity measurement after overnight incubation. A critical aspect of this multi-domain biosensor protein is that it is modular, potentially allowing new biosensors based on alternate human and animal NHRs to be generated by swapping NHR ligand binding domains^{45,46,47}. However, the system still relies on bacterial growth phenotypes for activity quantification, and thus requires a minimum overnight incubation to produce a sufficient signal. Also, this assay and other cell-based assays (i.e. bacterial, yeast, and mammalian) for detecting NHR-binding ligands can be affected by the presence of cytotoxic chemicals in samples and poor cellular uptake rates.

### SUMMARY

The present disclosure as disclosed in various embodiments is related to a bioassay for rapid screening and detection of bioactive ligands and screening for potential pharmaceuticals. In various embodiments are disclosed cell-free methods, systems, and kits for detecting bioactive ligands such as endocrine disrupting chemicals or nuclear hormone receptor modulators in a biological or environmental sample or for screening compounds for potential pharmaceutical application such as potential nuclear hormone receptor modulator pharmaceuticals.

In various embodiments are disclosed cell-free systems and methods of detecting compounds in a sample including the step of expressing biosensor proteins with a cell extract and in the presence of a sample, wherein the biosensor proteins during expressing bind compounds in the sample and are capable of generating detectable signals only when the biosensor proteins bind ligand during expressing.

In various embodiments are disclosed cell-free systems and methods of screening compounds for pharmaceutical applications including the step of expressing biosensor proteins with a cell extract and in the presence of a test compound, wherein the biosensor proteins are capable of generating detectable signals only when the biosensor proteins bind the test compound during expressing and the test compound is identified for a pharmaceutical application if it binds to the biosensor proteins.

In various embodiments are disclosed kits including an expression cassette including a polynucleotide encoding for a biosensor protein and a cell extract that is capable of being combined with at least the expression cassette and a sample or test compound in a cell-free reaction medium for expressing biosensor proteins. The kits of various embodiments can be used for detecting compounds in a sample or for screening compounds for pharmaceutical applications.

### BRIEF DESCRIPTION OF THE DRAWINGS

For a further understanding of the nature, objects, and advantages of the present invention, reference should be had to the following detailed description, read in conjunction with the following drawings, wherein:

FIGS. 1A, 1B, 1C, and 1D are the polynucleotide sequence encoding a thyroid receptor beta biosensor protein [SEQ ID NO: 1] of various embodiments.

FIGS. 2A and 2B are the amino acid sequence of a thyroid receptor β biosensor protein [SEQ ID NO: 2] of various embodiments having inactive amino- and carboxy-terminal intein splicing domains, a thyroid receptor β ligand-binding domain capable of binding to endocrine disrupting compounds, wherein the ligand-binding domain is linked to the inactive amino-terminal intein splicing domain on one end and to the inactive carboxy-terminal intein splicing domain on its other end, a reporter protein activably linked to the ligand-binding domain by at least one of the inactive intein terminal splicing domains, and a maltose binding protein linked to the ligand-binding domain by the other of the inactive intein terminal splicing domains.

FIGS. 3A, 3B, 3C, and 3D are the polynucleotide sequence encoding an estrogen receptor β biosensor protein [SEQ ID NO: 3] of various embodiments.

FIGS. 4A and 4B are the amino acid sequence of an estrogen receptor β biosensor protein [SEQ ID NO: 4] of various embodiments having inactive amino- and carboxy-terminal intein splicing domains, an estrogen receptor β ligand-binding domain capable of binding to endocrine disrupting compounds, wherein the ligand-binding domain is linked to the inactive amino-terminal intein splicing domain on one end and to the inactive carboxy-terminal intein splicing domain on its other end, a reporter protein activably linked to the ligand-binding domain by at least one of the inactive intein terminal splicing domains, and a maltose binding protein linked to the ligand-binding domain by the other of the inactive intein terminal splicing domains.

FIGS. 5A, 5B, and 5C show the polynucleotide sequence encoding a thyroid receptor beta biosensor protein [SEQ ID NO: 5] of various embodiments.

FIG. 6 shows the amino acid sequence of a thyroid receptor β biosensor protein [SEQ ID NO: 6] of various embodiments having inactive amino- and carboxy-terminal intein splicing domains, a thyroid receptor β ligand-binding domain capable of binding to endocrine disrupting compounds, wherein the ligand-binding domain is linked to the inactive amino-terminal intein splicing domain on one end and to the inactive carboxy-terminal intein splicing domain on its other end, a reporter protein activably linked to the ligand-binding domain by at least one of the inactive intein terminal splicing domains, and a chitin binding domain protein linked to the ligand-binding domain by the other of the inactive intein terminal splicing domains.

FIGS. 7A, 7B, and 7C show the polynucleotide sequence encoding an estrogen receptor β biosensor protein [SEQ ID NO: 7] of various embodiments.

FIG. 8 shows the amino acid sequence of an estrogen receptor β biosensor protein [SEQ ID NO: 8] of various embodiments having inactive amino- and carboxy-terminal intein splicing domains, an estrogen receptor β ligand-binding domain capable of binding to endocrine disrupting compounds, wherein the ligand-binding domain is linked to the inactive amino-terminal intein splicing domain on one end and to the inactive carboxy-terminal intein splicing domain on its other end, a reporter protein activably linked to the ligand-binding domain by at least one of the inactive intein terminal splicing domains, and a chitin binding domain protein linked to the ligand-binding domain by the other of the inactive intein terminal splicing domains.

FIG. 9 shows an example of the polypeptide construct of various embodiments transitioning to the biosensor protein in the presence or absence of a bioactive ligand. The polypeptide construct and biosensor protein as shown in FIG. 9 includes a Maltose Binding Protein (MBP) at its N-terminus, a mini-intein splicing domain with an inserted a receptor portion such as an NHR ligand binding domain, and a C-terminal reporter enzyme.

FIG. 10 shows example production yields of human thyroid receptor (hTRβ) biosensor proteins synthesized according to cell-free methods and system of various embodiments.

FIG. 11A shows an example of the polypeptide construct of various embodiments transitioning to the biosensor protein in the presence or absence of a bioactive ligand. The polypeptide construct and biosensor protein as shown in FIG. 11A includes a Chitin Binding Domain Protein (CBDP) at its N-terminus, a mini-intein splicing domain with an inserted a receptor portion such as an NHR ligand binding domain, and a C-terminal reporter enzyme.

FIG. 11B shows an example of the polypeptide construct of various embodiments transitioning to the biosensor protein in the presence or absence of a bioactive ligand. The polypeptide construct and biosensor protein as shown in FIG. 11B includes a reporter enzyme at its N-terminus, a mini-intein splicing domain with an inserted a receptor portion such as an NHR ligand binding domain, and a C-terminal CBDP.

FIG. 12 shows a schematic example describing cell-free methods and systems of various embodiments.

FIG. 13A shows an example dose response of hTRβ biosensor proteins of various embodiments in the presence of Tiratricol/3, 5, 3′-triiodothyroacetic acid (TRIAC).

FIG. 13B shows an example dose response curve of hTRβ biosensor proteins of various embodiments in the presence of T3 hormones (triangles) and E2 hormones (squares).

FIG. 13C shows examples of the half-maximal effective concentration (EC₅₀), slope factor (k), Z′ factor, signal to noise ratio (S/N), and signal to background ratio (S/B) for the responses of the hTRβ biosensor proteins of various embodiments against TRIAC, T3, and E2.

FIG. 13D shows an example dose-response graph of the cell-free methods and systems of various embodiments with biosensor proteins and lyophilized cell extract in the presence of TRIAC.

FIG. 13E shows an example statistical analysis results of the cell-free methods and systems of various embodiments with biosensor proteins and lyophilized cell extract in the presence of TRIAC.

FIG. 14 shows example dose responses of hTRβ biosensor proteins of various embodiments with MBP as shown in FIG. 9 (Black) and with CBDP as shown in FIG. 10 (Gray) the presence of TRIAC.

FIG. 15 shows example dose responses of hTRβ biosensor proteins of various embodiments with a polyhistidine-tag and including MBP as shown in FIG. 9 (Black) or CBDP as shown in FIG. 10 (Gray) the presence of T3 hormones.

FIG. 16 shows an example of the protein production capability of the cell-free methods and system of various embodiments in the presence of environmental and human samples.

FIGS. 17A and 17B show examples of dose-response graph and statistical analysis results of the cell-free methods and system of various embodiments in the presence of TRIAC and 40% by volume raw sewage.

FIG. 18A shows an example of an estrogen receptor β biosensor construct of various embodiments transitioning to an estrogen receptor β biosensor protein in the presence of a hERβ-specific ligand. The polypeptide construct and biosensor protein as shown in FIG. 18A includes an MBP at its N-terminus, a mini-intein splicing domain with an inserted estrogen receptor β ligand binding domain, and a C-terminal reporter enzyme.

FIGS. 18B and 18C show schematic examples describing cell-free methods and system of various embodiments in the presence or absence of hERβ-specific ligands.

FIG. 19A shows an example of an estrogen receptor β biosensor construct of various embodiments transitioning to an estrogen receptor β biosensor protein in the presence of a hERβ-specific ligand. The polypeptide construct and biosensor protein as shown in FIG. 19A includes a CBDP at its N-terminus, a mini-intein splicing domain with an inserted estrogen receptor β ligand binding domain, and a C-terminal reporter enzyme.

FIG. 19B shows an example of an estrogen receptor β biosensor construct of various embodiments transitioning to an estrogen receptor β biosensor protein in the presence of a hERβ-specific ligand. The polypeptide construct and biosensor protein as shown in FIG. 19B includes a reporter enzyme at its N-terminus, a mini-intein splicing domain with an inserted estrogen receptor β ligand binding domain, and a C-terminal CBDP.

FIG. 20 shows example production yields of estrogen receptor β biosensor proteins synthesized according to cell-free methods and system of various embodiments.

FIGS. 21A, 21B, 21C, and 21D show example does response graphs of estrogen receptor β biosensor proteins of various embodiments in the presence of E2 hormone, Diarylpropionitrile/2,3-bis(4-Hydroxyphenyl)-propionitrile (DPN), Bisphenol A/4,4′-(propane-2,2-diyl)diphenol (BPA), and TRIAC (negative control, normalized based on the BPA maximum response).

FIG. 21E shows example statistical analysis results of the half-maximal EC₅₀, k, S/N, S/B, and limit of detection (LOD) for the responses of the estrogen receptor β biosensor proteins of various embodiments in the presenCe of E2 hormone, DPN, BPA, and TRAIC.

FIG. 22 shows example dose responses of the estrogen receptor β biosensor proteins of various embodiments with MBP as shown in FIG. 18A (Black solid), with CBDP as shown in FIG. 19 (Gray solid), with MBP and a peptide linker (IGS) (Black dots), and with CBDP and a peptide linker (IGS) (Gray dots) in the presence of E2.

FIG. 23 shows an example effect of urea on the cell-free methods and system of various embodiments.

FIGS. 24A and 24B show example effects of RNase inhibitor on the C-terminal reporter enzyme (e.g. green fluorescent protein) of the cell-free methods and system of various embodiments in urine (FIG. 24A) and blood (FIG. 24B).

FIGS. 25A and 25B show example dose-response graphs for the estrogen receptor β biosensor proteins of various embodiments in 20% by volume blood (FIG. 25B) and 10% by volume urine (FIG. 25A).

FIG. 25C shows examples statistical analysis results of the estrogen receptor β biosensor proteins of various embodiments in 20% by volume blood and 10% by volume urine.

### BRIEF DESCRIPTION OF THE SEQUENCES

SEQ ID NO: 1 sets forth a polynucleotide sequence encoding a thyroid receptor β biosensor protein including a maltose binding protein.

SEQ ID NO: 2 sets forth an amino acid sequence of a thyroid receptor β biosensor protein including a maltose binding protein.

SEQ ID NO: 3 sets forth a polynucleotide sequence encoding an estrogen receptor β biosensor protein including a maltose binding protein.

SEQ ID NO: 4 sets forth an amino acid sequence of an estrogen receptor β biosensor protein including a maltose binding protein.

SEQ ID NO: 5 sets forth a polynucleotide sequence encoding a thyroid receptor β biosensor protein including a chitin binding domain protein.

SEQ ID NO: 6 sets forth an amino acid sequence of a thyroid receptor β biosensor protein including a chitin binding domain protein.

SEQ ID NO: 7 sets forth a polynucleotide sequence encoding an estrogen receptor β biosensor protein including a chitin binding domain protein.

SEQ ID NO: 8 sets forth an amino acid sequence of an estrogen receptor β biosensor protein including a chitin binding domain protein.

### DETAILED DESCRIPTION

As required, detailed embodiments of the present invention are disclosed herein; however, it is to be understood that the disclosed embodiments are merely exemplary of the invention that may be embodied in various and alternative forms. The figures are not necessarily to scale; some features may be exaggerated or minimized to show details of particular components. Therefore, specific structural and functional details disclosed herein are not to be interpreted as limiting, but merely as a representative basis for teaching one skilled in the art to variously employ the present invention.

Except in the examples, or where otherwise expressly indicated, all numerical quantities in this description indicating amounts of material or conditions of reaction or use are to be understood as modified by the word “about” in describing the broadest scope of the invention. Practice within the numerical limits stated is generally preferred. The first definition of an acronym or other abbreviation applies to all subsequent uses herein of the same abbreviation and applies mutatis mutandis to normal grammatical variations of the initially defined abbreviation; and, unless expressly stated to the contrary, measurement of a property is determined by the same technique as previously or later referenced for the same property.

Unless indicated otherwise, all technical and scientific terms used herein have the same meaning as commonly understood by one of ordinary skill in the art to which this invention belongs.

Throughout this application, where publications are referenced, the disclosures of these publications in their entireties are hereby incorporated by reference into this application to more fully describe the state of the art to which this invention pertains.

It is also to be understood that this invention is not limited to the specific embodiments and methods described below, as specific components or conditions may, of course, vary. Furthermore, the terminology used herein is used only for the purpose of describing particular embodiments of the present invention and is not intended to be limiting in any way.

It must also be noted that, as used in the specification and the appended claims, the singular form “a,” “an,” and “the” comprise plural referents unless the context clearly indicates otherwise. For example, reference to a component in the singular is intended to comprise a plurality of components.

The term “or” is understood to mean “and/or”.

The term “comprising” is synonymous with “including,” “having,” “containing,” or “characterized by.” These terms are inclusive and open-ended and do not exclude additional, unrecited elements or method steps.

The phrase “consisting of” excludes any element, step, or ingredient not specified in the claim. When this phrase appears in a clause of the body of a claim, rather than immediately following the preamble, it limits only the element set forth in that clause; other elements are not excluded from the claim as a whole.

The phrase “consisting essentially of” limits the scope of a claim to the specified materials or steps, plus those that do not materially affect the basic and novel characteristic(s) of the claimed subject matter.

The terms “comprising”, “consisting of”, and “consisting essentially of” can be alternatively used. When one of these three terms is used, the presently disclosed and claimed subject matter can include the use of either of the other two terms.

As used herein, “kit” means a collection of at least two components constituting the kit. Together, the components constitute a functional unit for a given purpose. Individual member components may be physically packaged together or separately.

The terms “polynucleotide”, “nucleotide”, “nucleotide sequence”, “nucleic acid” and “oligonucleotide” are used interchangeably in this disclosure. They refer to a polymeric form of nucleotides of any length, either deoxyribonucleotides or ribonucleotides, or analogs thereof. Polynucleotides may have any three-dimensional structure, and may perform any function, known or unknown. The following are non-limiting examples of polynucleotides: single-, double-, or multi-stranded DNA or RNA, cDNA, DNA-RNA hybrids, or a polymer comprising purine and pyrimidine bases or other natural, chemically or biochemically modified, non-natural, or derivatized nucleotide bases. The terms “polynucleotide” and “nucleic acid” should be understood to include, as applicable to the embodiment being described, single-stranded (such as sense or antisense) and double-stranded polynucleotides A polynucleotide may comprise one or more modified nucleotides, such as methylated nucleotides and nucleotide analogs. If present, modifications to the nucleotide structure may be imparted before or after assembly of the polymer. The sequence of nucleotides may be interrupted by non-nucleotide components. A polynucleotide may be further modified after polymerization, such as by conjugation with a labeling component.

The terms “amino acid sequence” or “amino acid” refers to a list of abbreviations, letters, characters or words representing amino acid residues. The amino acid abbreviations used herein are conventional one letter codes for the amino acids and are expressed as follows: A, alanine; C, cysteine; D aspartic acid; E, glutamic acid; F, phenylalanine; G, glycine; FI histidine; I isoleucine; K, lysine; L, leucine; M, methionine; N, asparagine; P, proline; Q, glutamine; R, arginine; S, serine; T, threonine; V, valine; W, tryptophan; Y, tyrosine.

The terms “sequence identity” or “identity” refers to a specified percentage of residues in two nucleic acid or amino acid sequences that are identical when aligned for maximum correspondence over a specified comparison window, as measured by sequence comparison algorithms or by visual inspection. When sequences differ in conservative substitutions, the percent sequence identity may be adjusted upwards to correct for the conservative nature of the substitution. Sequences that differ by such conservative substitutions are said to have “sequence similarity” or “similarity.” Means for making this adjustment are well known to those of skill in the art. Typically this involves scoring a conservative substitution as a partial rather than a full mismatch, thereby increasing the percentage sequence identity.

The terms “peptide” or “protein” as used herein refers to any peptide, oligopeptide, polypeptide, gene product, expression product, or protein. A peptide is comprised of consecutive amino acids. The term “peptide” encompasses naturally occurring or synthetic molecules.

The terms “construct”, “cassette”, “expression cassette”, “plasmid”, “vector”, or “expression vector” is understood to mean a recombinant nucleic acid, generally recombinant DNA, which has been generated for the purpose of the expression or propagation of a nucleotide sequence(s) of interest, or is to be used in the construction of other recombinant nucleotide sequences.

The term “promoter” or “promoter polynucleotide” is understood to mean a regulatory sequence/element or control sequence/element that is capable of binding/recruiting a RNA polymerase and initiating transcription of sequence downstream or in a 3′ direction from the promoter. A promoter can be, for example, constitutively active or always on or inducible in which the promoter is active or inactive in the presence of an external stimulus. Example of promoters include T7 promoters or U6 promoters.

The term “operably linked” can mean the positioning of components in a relationship which permits them to function in their intended manner. For example, a promoter can be linked to a polynucleotide sequence to induce transcription of the polynucleotide sequence.

A fusion protein is a polypeptide generated by expression of a polynucleotide coding sequence that is made up of at least two in-frame coding regions that do not naturally occur together.

The term “intein” refers to an in-frame intervening sequence in a protein. An intein can catalyze its own excision from the protein through a post-translational protein splicing process to yield the free intein and a mature protein. As used herein, “intein” encompasses mini-inteins, modified or mutated inteins, and split inteins. The term “inactivated intein”, “inactive intein”, “inactive intein terminal splicing domains”, “inactive amino- and carboxy-terminal intein splicing domains”, “inactive amino-terminal intein splicing domain”, or “inactive carboxy-terminal intein splicing domain” are an intein or part of an intein having an amino acid sequence that has been altered such that the ability of the intein to cleave and splice a polypeptide in which the intein occurs has been substantially eliminated. For example, inactivated intein can be modified to alter their splicing motifs eliminated cleavage or splicing of an endonuclease domain or a receptor portion replacing the endonuclease domain. Modification of the splicing motifs can include point mutation of a nucleotide or nucleotides within the splicing motifs.

The term “receptor portion” refers to at least a portion of a receptor such as binding sites that can bind or be bound to a ligand such as a bioactive ligand. A receptor portion can also include multiple binding sites or a full length receptor.

A ligand-binding domain of a protein is a part of a protein having chemical properties and a conformation in the native protein that confers to the domain the ability to bind with a compound (normally a compound much smaller than the protein) with the specificity normally associated with binding between a protein receptor and its corresponding ligand.

The term “cell extract” is understood to mean any preparation comprising the components of a protein synthesis machinery, usually a prokaryotic cell extract (i.e. bacterial cell extract) or eukaryotic cell extract (i.e. fungal cell extract such as yeast or mammalian cell extract), wherein such components are capable of expressing a nucleic acid encoding a desired protein. Thus, a prokaryotic cell extract or eukaryotic cell extract can include components/machinery that are capable of translating messenger ribonucleic acid (mRNA) encoding a desired protein, and optionally comprises components that are capable of transcribing DNA encoding a desired protein. Such components for the prokaryotic cell extract can include, for example, DNA-directed RNA polymerase (RNA polymerase), any transcription activators that are required for initiation of transcription of DNA encoding the desired protein, transfer ribonucleic acids (tRNAs), aminoacyl-tRNA synthetases, 70S ribosomes, N₁₀-formyltetrahydrofolate, formylmethionine-tRNAfMet synthetase, peptidyl transferase, initiation factors such as IF-1, IF-2 and IF-3, elongation factors such as EF-Tu, EF-Ts, and EF-G, release factors such as RF-1, RF-2, and RF-3, and the like. Components for the eukaryotic cell extract can include, for example, the components listed in paragraphs [0027]-[0045] of U.S. Patent Application Publication No. 2011/0300575, which is incorporated in its entirety by reference.

The terms “cell-free” or “without viable cells” is understood to mean any type of system capable of synthesizing proteins in vitro in the absence of viable cells. Examples can include cell-free protein synthesis system derived from bacterial cell extracts such as extracts from E. coli or mammalian cell. The terms “cell-free” or “without viable cells” can alternatively be understood to mean including viable cells or trace amounts of viable cells, where the biosensor protein is synthesized outside of or extracellularly from the viable cells.

The present disclosure as disclosed in various embodiments is related to a bioassay for rapid screening and detection of bioactive ligands and screening for potential pharmaceuticals. In various embodiments are disclosed cell-free methods, systems, and kits for detecting bioactive ligands such as endocrine disrupting chemicals or nuclear hormone receptor modulators in a biological or environmental sample or for screening compounds for potential pharmaceutical application such as potential nuclear hormone receptor modulator pharmaceuticals.

In various embodiments are disclosed cell-free systems and methods of detecting compounds in a sample including the step of expressing biosensor proteins with a cell extract and in the presence of a sample, wherein the biosensor proteins during expressing bind compounds in the sample and are capable of generating detectable signals only when the biosensor proteins bind ligand during expressing. The compound of various embodiments can be a chemical or a ligand such as a bioactive ligand.

In various embodiments are disclosed cell-free systems and methods of detecting bioactive ligands in a biological or environmental sample including the steps of: providing an expression cassette including a polynucleotide encoding for a biosensor protein comprising: inactive amino- and carboxy-terminal intein splicing domains, a receptor portion capable of binding a bioactive ligand to form a receptor-ligand complex, wherein the at least a portion of a receptor is linked to the inactive amino-terminal intein splicing domain on one end and to the inactive carboxy-terminal intein splicing domain on its other end, and a reporter protein activably linked to the receptor portion by at least one of the inactive intein terminal splicing domains; synthesizing biosensor proteins in the presence of a biological or environmental sample and from the expression cassette with a cell extract without viable cells, wherein the biosensor proteins have a properly folded, biologically active conformation when the receptor portions bind bioactive ligands in the biological or environmental sample during synthesis; and detecting the presence of bioactive ligands in the biological or environmental sample, wherein the reporter proteins of the biosensor proteins having the properly folded, biologically active conformation are capable of generating signals for detection. The biosensor protein of various embodiments also has an improperly folded, biologically inactive conformation when the receptor portions do not bind bioactive ligands during synthesis, where the reporter proteins of the biosensor proteins have an improperly folded, biologically inactive conformation are unable of generating signals for detection.

In various embodiments are disclosed cell-free systems and methods of screening compounds for pharmaceutical applications including the step of expressing biosensor proteins with a cell extract and in the presence of a test compound, wherein the biosensor proteins are capable of generating detectable signals only when the biosensor proteins bind the test compound during expressing and the test compound is identified for a pharmaceutical application if it binds to the biosensor proteins. In various embodiments are disclosed methods and systems for high-throughput screening of potential nuclear hormone receptor modulator pharmaceutical using a biosensor protein. The test compound can be identified as an antagonist to ligand-binding to or an agonist of a receptor if the test compound binds to the biosensor proteins. Antagonist can include various forms of inhibition such as competitive, non-competitive, and uncompetitive. The pharmaceutical applications of various embodiments can include nuclear hormone modulators, where the test compound is identified as a nuclear hormone modulator if it binds to the biosensor proteins.

In various embodiments are disclosed cell-free systems and methods of screening compounds for pharmaceutical applications including the steps of: providing an expression cassette including a polynucleotide encoding for a biosensor protein comprising: inactive amino- and carboxy-terminal intein splicing domains, a receptor portion capable of binding a bioactive ligand to form a receptor-ligand complex, wherein the at least a portion of a receptor is linked to the inactive amino-terminal intein splicing domain on one end and to the inactive carboxy-terminal intein splicing domain on its other end, and a reporter protein activably linked to the receptor portion by at least one of the inactive intein terminal splicing domains; synthesizing biosensor proteins in the presence of a test compound and from the expression cassette with a cell extract without viable cells, wherein the biosensor proteins have a properly folded, biologically active conformation when the receptor portions bind bioactive ligands in the biological or environmental sample during synthesis; and detecting the presence of bioactive ligands in the biological or environmental sample, wherein the reporter proteins of the biosensor proteins having the properly folded, biologically active conformation are capable of generating signals for detection. The biosensor protein of various embodiments also has an improperly folded, biologically inactive conformation when the receptor portions do not bind bioactive ligands during synthesis, where the reporter proteins of the biosensor proteins have an improperly folded, biologically inactive conformation are unable of generating signals for detection. In various embodiments, the cell-free systems and methods of screening compounds for pharmaceutical applications can further include the step of identifying the test compound for the pharmaceutical application if it binds to the biosensor proteins, where a signal can be detected.

In various embodiments, the method and systems reduce the assay duration of existing whole-cell, transcriptionally activated biosensors of hormone receptor modulators from 18 hours to about 20 minutes. The method and systems of various embodiments can be completed within about 20 minutes, 25 minutes, 30 minutes, 35 minutes, 40 minutes, 45 minutes, 50 minutes, 55 minutes, 60 minutes, 65 minutes, 70 minutes, 75 minutes, 80 minutes, 85 minutes, 90 minutes, 95 minutes, 100 minutes, 105 minutes, 110 minutes, 115 minutes, and 120 minutes. In various embodiments, the time to complete the methods and systems of various embodiments is a range between any two times listed above.

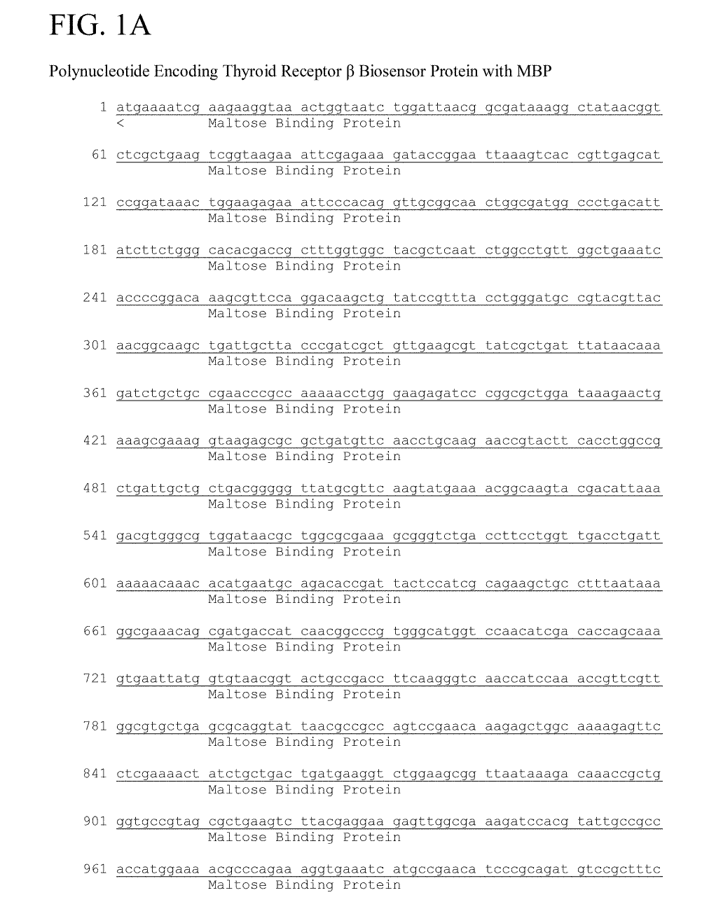

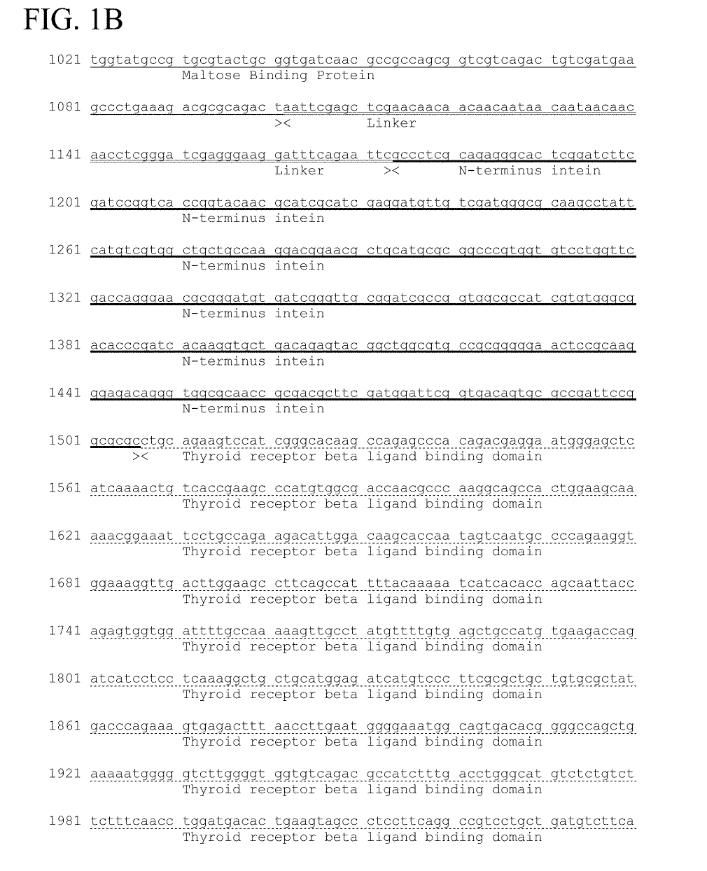

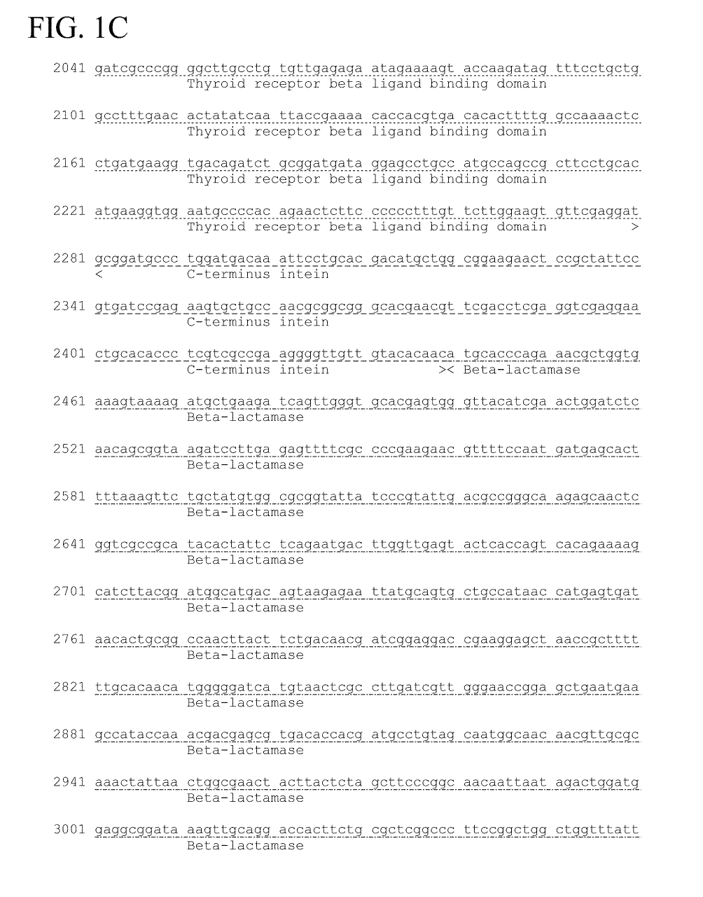

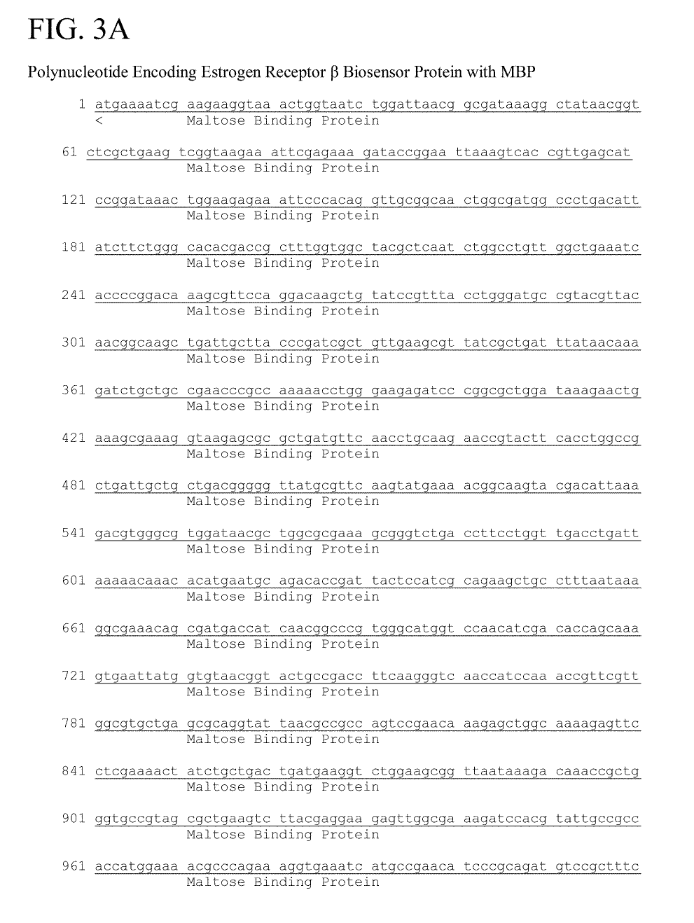

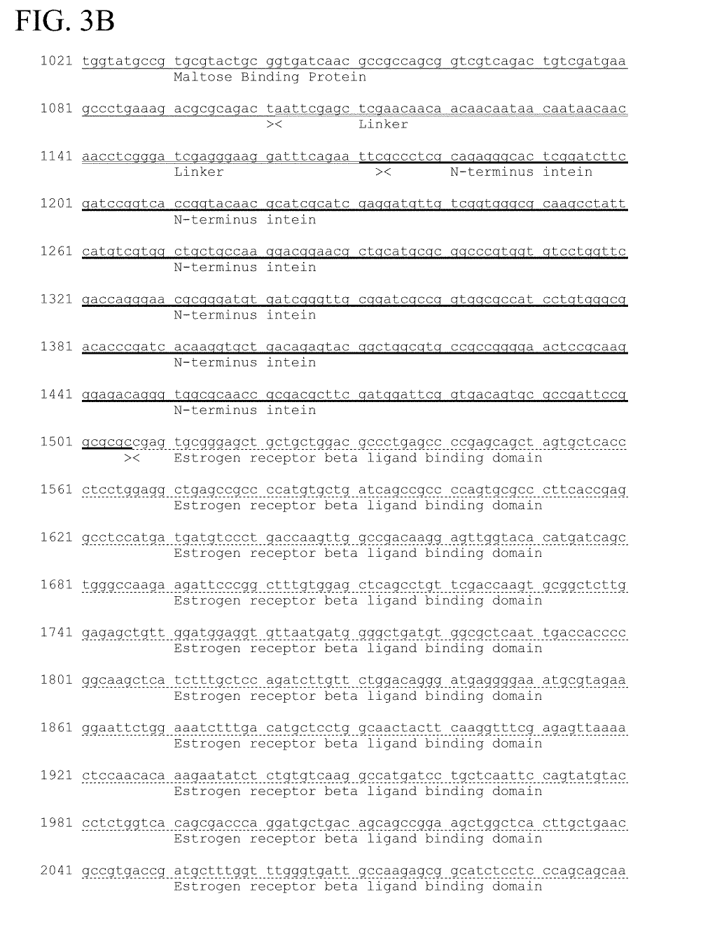

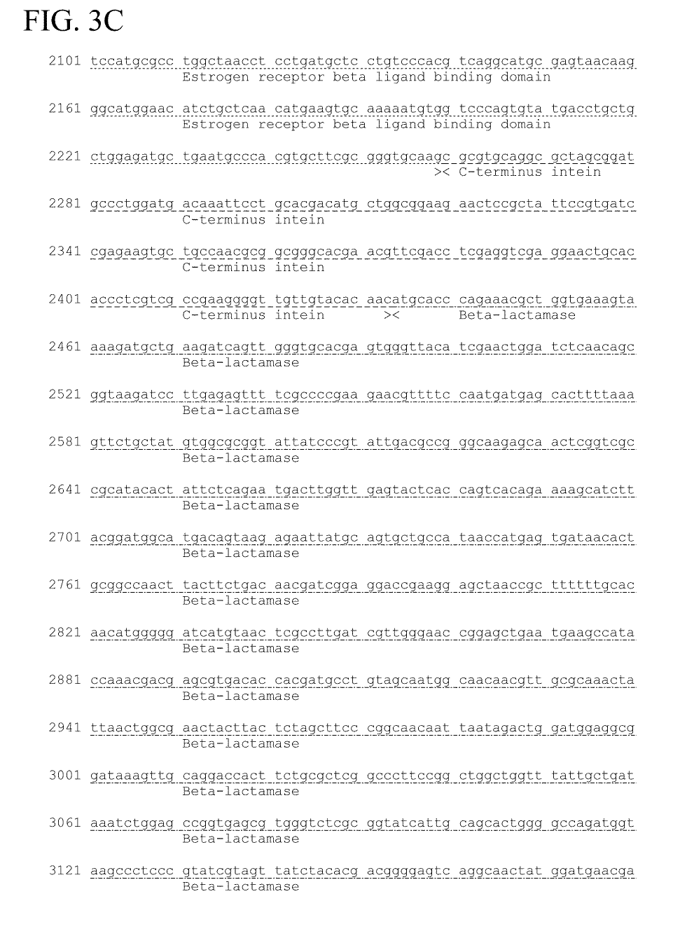

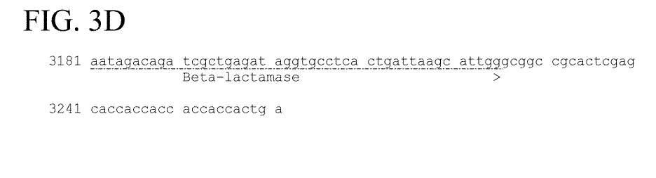

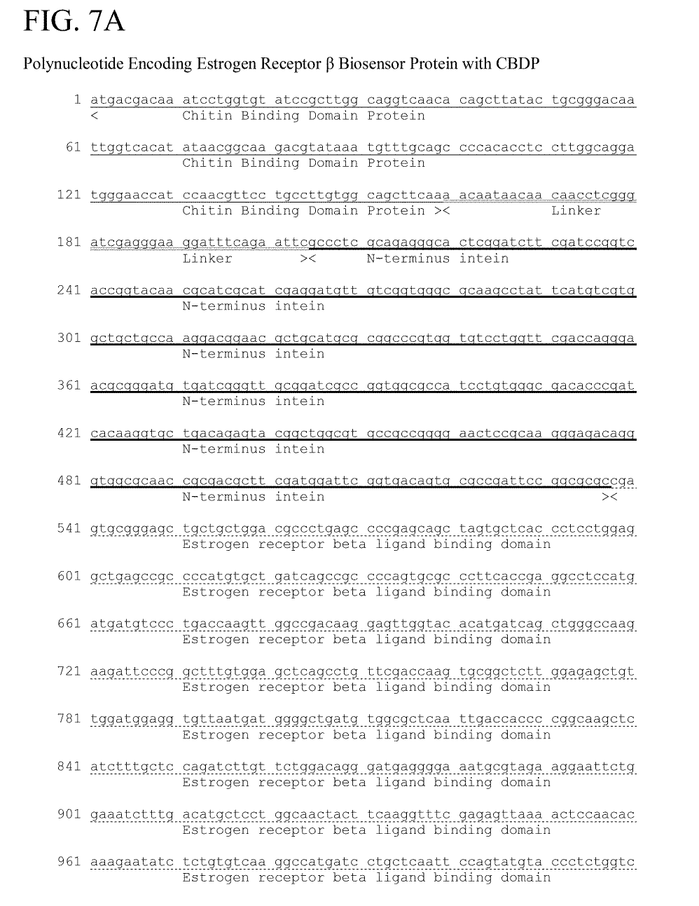

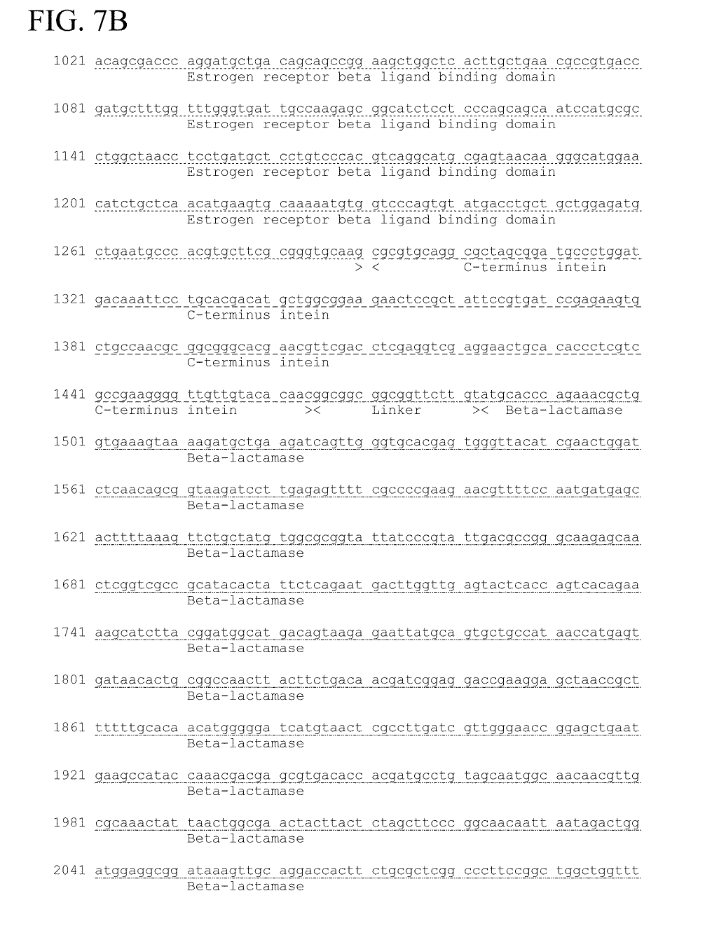

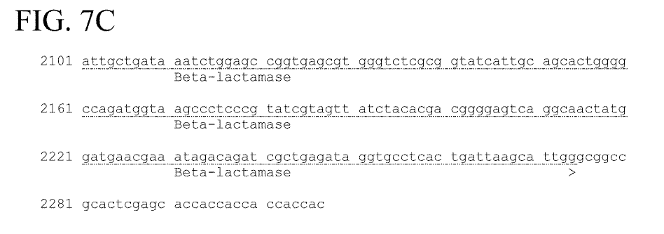

Examples of the polynucleotide of various embodiments include SEQ ID NO: 1 as shown in FIGS. 1A-1D; SEQ ID NO: 3 as shown in FIGS. 3A-3D; SEQ ID NO: 5 as shown in FIGS. 5A-5C; and SEQ ID NO: 7 as shown in FIGS. 7A-7C.

In various embodiments, the polynucleotide of various embodiments includes a sequence of nucleotide(nt) 1 to nt 1101, nt 1102 to nt 1173, nt 1174 to nt 1506, nt 1507 to nt 2280, nt 2281 to nt 2439, nt 2440 to nt 3267, or combination thereof of polynucleotide SEQ ID NO: 1. As shown in FIGS. 1A-1D, the above sequences encode for different elements of the biosensor protein of various embodiments.

In various embodiments, the polynucleotide of various embodiments includes a sequence of nt 1 to nt 1101, nt 1102 to nt 1173, nt 1174 to nt 1506, nt 1507 to nt 2259, nt 2260 to nt 2433, nt 2434 to nt 3261, or combination thereof of polynucleotide SEQ ID NO: 3. As shown in FIGS. 3A-3D, the above sequences encode for different elements of the biosensor protein of various embodiments.

In various embodiments, the polynucleotide of various embodiments includes a sequence of nt 1 to nt 159, nt 160 to nt 204, nt 206 to nt 537, nt 538 to nt 1311, nt 1312 to nt 1485, nt 1486 to nt 1503, nt 1504 to nt 2259, or combination thereof of polynucleotide SEQ ID NO: 5. As shown in FIGS. 5A-5C, the above sequences encode for different elements of the biosensor protein of various embodiments.

In various embodiments, the polynucleotide of various embodiments includes a sequence of nt 1 to nt 159, nt 160 to nt 204, nt 205 to nt 537, nt 538 to nt 1311, nt 1312 to nt 1485, nt 1486 to nt 1503, nt 1504 to nt 2295, or combination thereof of polynucleotide SEQ ID NO: 5. As shown in FIGS. 5A-5C, the above sequences encode for different elements of the biosensor protein of various embodiments.

In various embodiments, the polynucleotide of various embodiments includes a sequence of nt 1 to nt 159, nt 160 to nt 204, nt 205 to nt 537, nt 538 to nt 1290, nt 1291 to nt 1464, nt 1465 to nt 1482, nt 1483 to nt 2274, or combination thereof of polynucleotide SEQ ID NO: 7. As shown in FIGS. 7A-7C, the above sequences encode for different elements of the biosensor protein of various embodiments.

The expression cassette of various embodiments can further include a regulatory/promoter polynucleotide sequence operably linked to the polynucleotide encoding for the biosensor protein. Examples of promoters include, but are not limited to, T7 promoters or U6 promoters. The expression cassette of various embodiments can include, for example, plasmids, constructs, cassettes, vectors, or expression vectors.

In various embodiments, the inactive intein terminal splicing domains are unable to splice the receptor portion. A database of known inteins is maintained by New England Biolabs (www.inteins.com, accessed on 14 Nov. 2017), which discloses various conserved intein features and splicing motifs. The N- and C-terminal intein motifs includes sequences such as blocks A, N2, B, N4, F, and G, wherein the different blocks can have conserved amino acids or be next to conserved residues in an extein protein that effect the splicing function of the inteins. For example, block A can have a conserved amino acid (e.g. C,S), block B can have a conserved amino acid sequence such as TxxH, and block G can have a conserved amino acid (e.g. N,H) or be next to a conserved amino acid in the extein (e.g. C,S,T). In various embodiments, the inactive terminal splicing domains are modified to not include conserved amino acids or conserved amino acid sequences or are adjacent to a non-conserved amino acid.

In various embodiments, the polynucleotide sequence encoding for the inactive amino-terminal intein splicing domain or carboxy-terminal intein splicing domain can have 95%, 96%, 97%, 98%, 99% or 100% sequence identity or sequence similarity of the part of polynucleotides SEQ ID NO: 1, SEQ ID NO: 3, SEQ ID NO: 5, and SEQ ID NO: 7 encoding for the inactive amino-terminal intein splicing domain and carboxy-terminal intein splicing domain. In various embodiments, the sequence identity percentage or sequence similarity percentage is a range between any two sequence identity percentages listed above.

In various embodiments, the amino acid sequence of the inactive amino-terminal intein splicing domain or carboxy-terminal intein splicing domain can have 95%, 96%, 97%, 98%, 99% or 100% sequence identity or sequence similarity of the part of amino acid SEQ ID NO: 2, SEQ ID NO: 4, SEQ ID NO: 6, and SEQ ID NO: 8 of the inactive amino-terminal intein splicing domain and carboxy-terminal intein splicing domain. In various embodiments, the sequence identity percentage or sequence similarity percentage is a range between any two sequence identity percentages listed above.

In various embodiments, the receptor portion includes a binding site capable of binding to the bioactive ligand. The receptor portion of various embodiments can also include a plurality of binding sites or a full-length receptor that is capable of binding the bioactive ligand. Examples of receptor portion include, but are not limited to, binding sites or ligand-binding domains of nuclear hormone receptors, cell-surface receptors, G-protein coupled receptors, or cytokine receptors. The receptor portion of various embodiments can be derived from various sources include, for example, mammals such as humans.

In various embodiments, the biosensor protein further includes a solubility enhancer capable of increasing the solubility of the biosensor protein, where the biosensor protein with the solubility enhancer has a greater solubility than an otherwise identical biosensor protein devoid of the solubility enhancer. The solubility enhancer of various embodiments is linked to the at least a portion of a receptor by the other of the inactive intein terminal splicing domains. Examples of solubility enhancers include, but are not limited to, maltose-binding proteins or chitin binding domain proteins.

In various embodiments, the reporter protein is an enzymatic or fluorescent reporter protein and the reporter proteins of the biosensor proteins having the properly folded, biologically active conformation generate visual signals in the presence of a substrate or stimulus. Examples of the enzymatic reporter protein can include, but are not limited to, beta-lactamase enzymes, β-galactosidase, or luciferase and examples of substrates can include, but are not limited to, nitrocefin, 5-bromo-4-chloro-3-indolyl-β-D-galactopyranoside, or luciferin. Examples of fluorescent reporter proteins can include, but are not limited to, green fluorescent protein, red fluorescent protein, or different variants of the green or red fluorescent proteins and examples of stimuli can include apparatus capable of emitting excitation wavelengths for the fluorescent proteins.

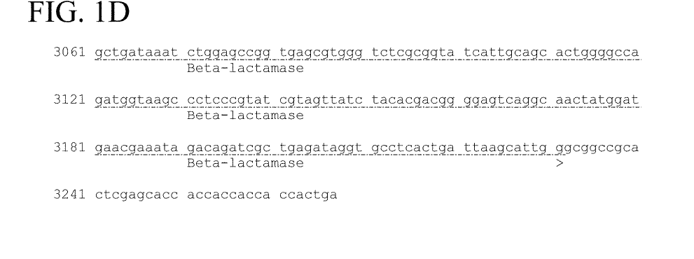

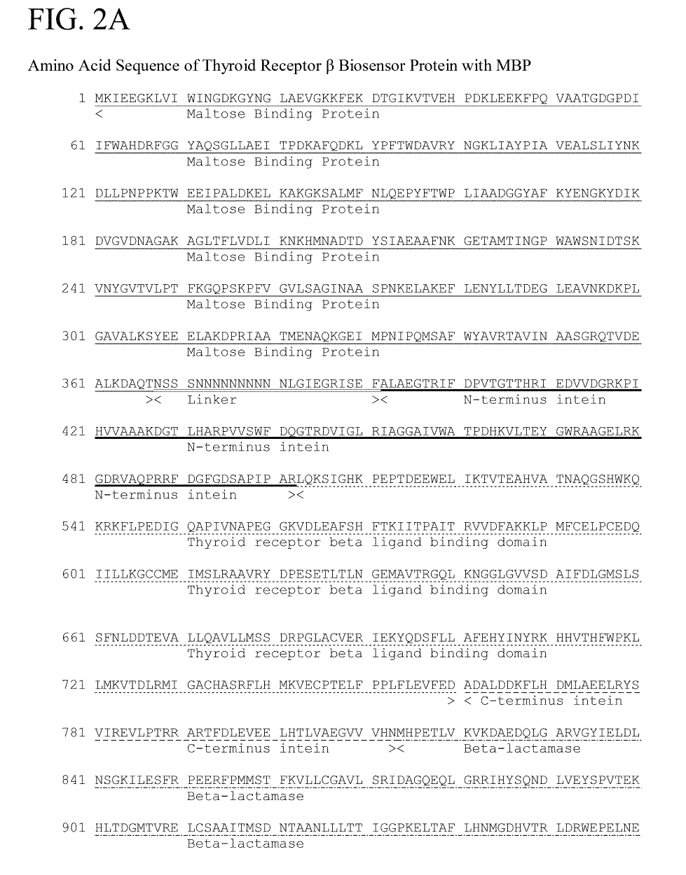

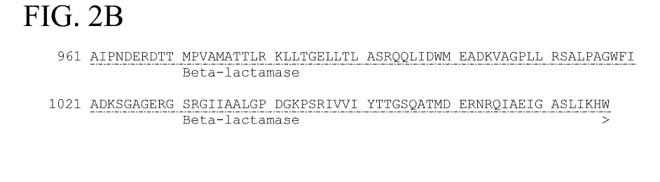

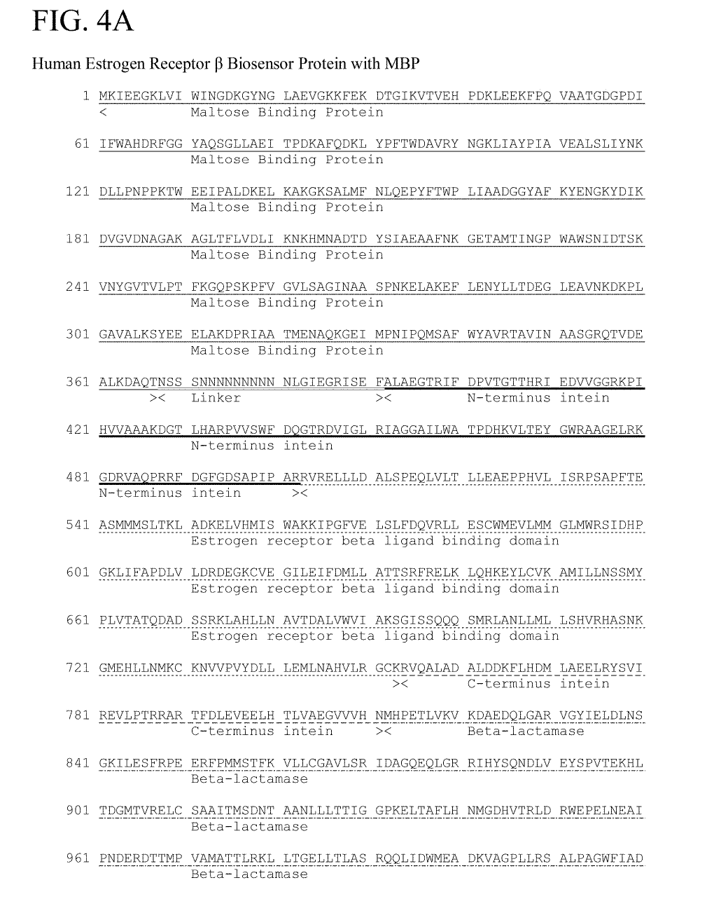

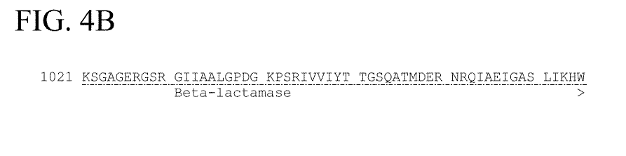

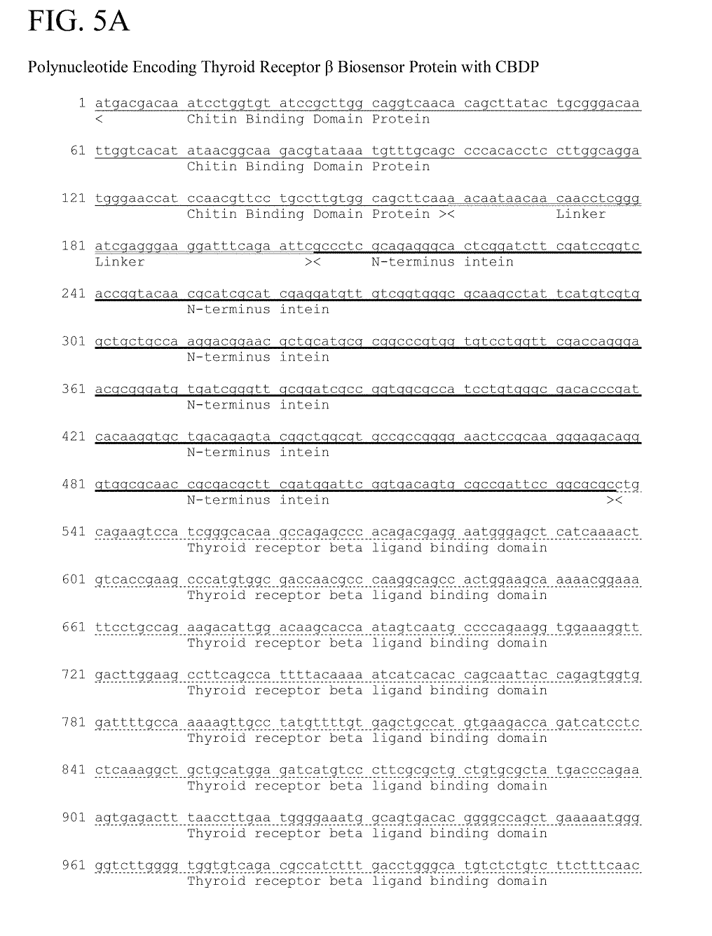

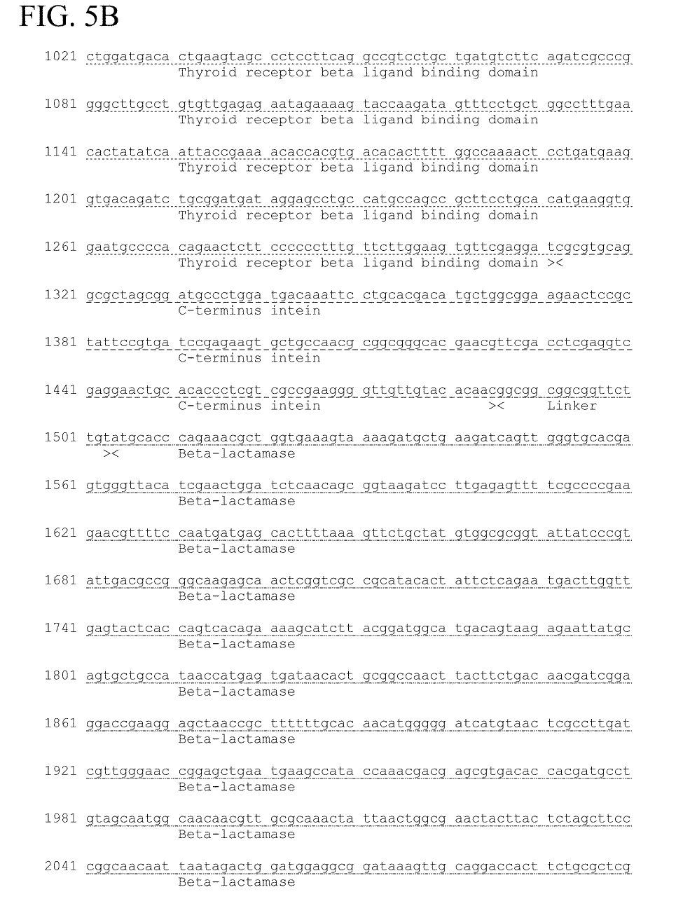

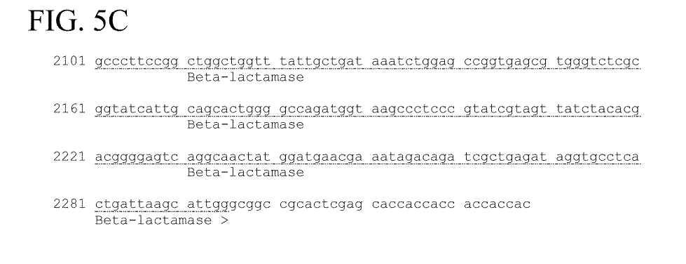

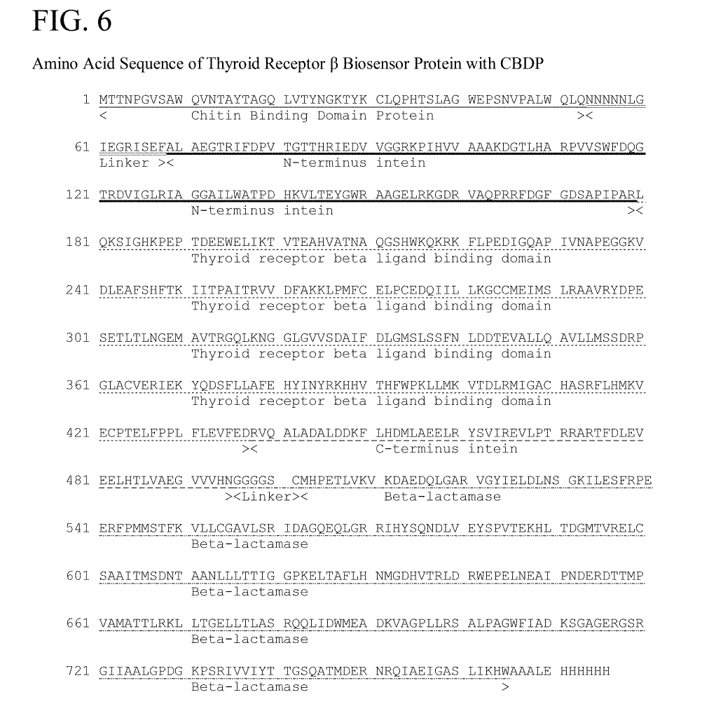

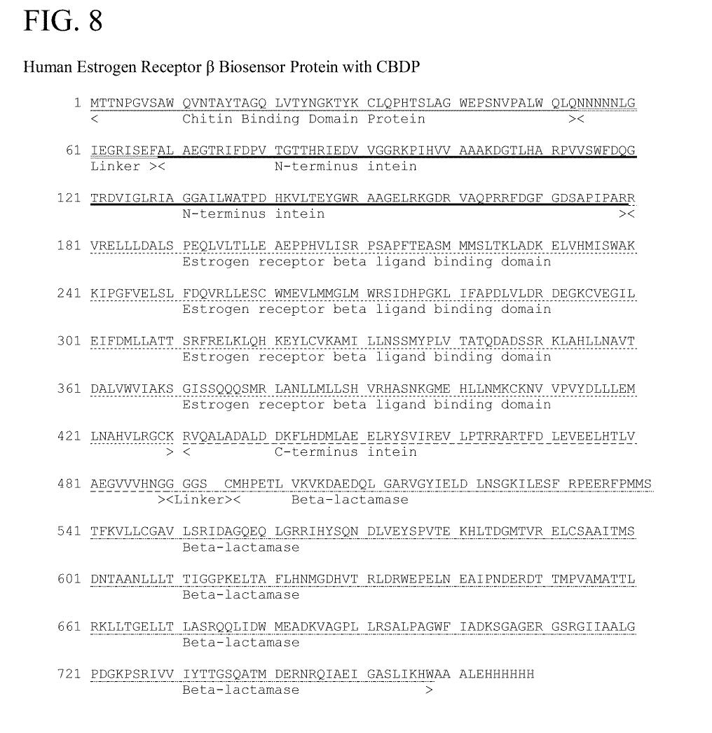

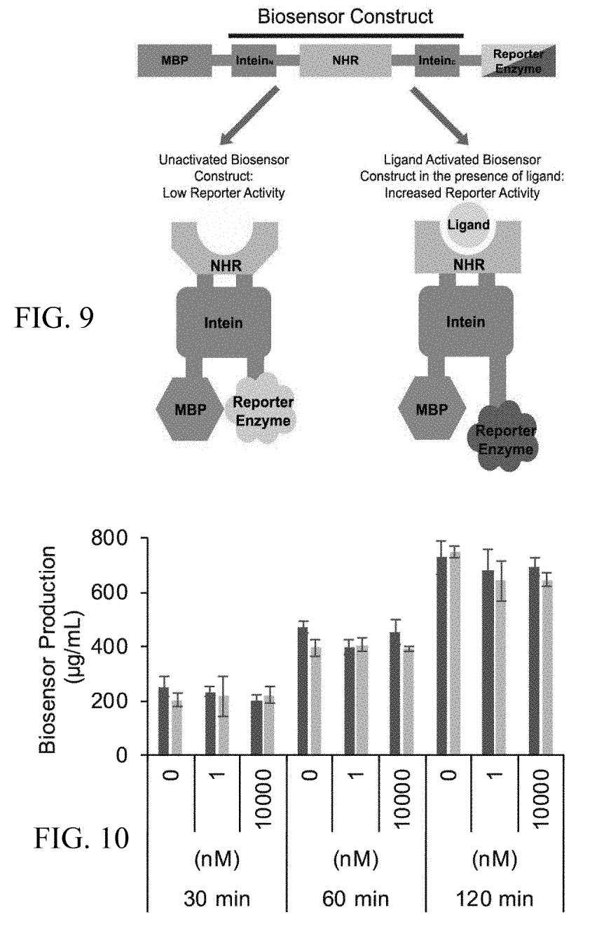

In different examples: the polynucleotide of various embodiments generally including SEQ ID NO: 1, which generally encodes for the polypeptide construct of SEQ ID NO: 2 as shown in FIGS. 2A and 2B; the polynucleotide of various embodiments generally including SEQ ID NO: 3 that generally encodes for the polypeptide construct of SEQ ID NO: 4 shown in FIGS. 4A and 4B; the polynucleotide of various embodiments generally including SEQ ID NO: 5, which generally encodes for the polypeptide construct of SEQ ID NO: 6 shown in FIG. 6; and the polynucleotide of various embodiments generally including SEQ ID NO: 7, which generally encodes for the polypeptide construct of SEQ ID NO: 8 shown in FIG. 8. The polypeptide constructs of SEQ ID NO: 2; SEQ ID NO: 4; SEQ ID NO: 6; and SEQ ID NO: 8 are capable of transitioning/folding into biosensor proteins of various embodiments as shown in FIGS. 9, 11A, 11B, 18A, 19A, and 19B.

In various embodiments, the biosensor protein of various embodiments includes a sequence of amino acid (aa) 1 to aa 367, aa 368 to aa 391, aa 392 to aa 502, aa 503 to aa 760, aa 761 to aa 813, aa 814 to aa 1077, or combinations thereof of SEQ ID NO: 2. As shown in FIGS. 2A-2B, the above sequences are for different elements of the biosensor protein of various embodiments.

In various embodiments, the biosensor protein of various embodiments includes a sequence of aa 1 to aa 367, aa 368 to aa 391, aa 392 to aa 502, aa 503 to aa 753, aa 754 to aa 811, aa 812 to aa 1075, or combinations thereof of SEQ ID NO: 4. As shown in FIGS. 4A-4B, the above sequences are for different elements of the biosensor protein of various embodiments.

In various embodiments, the biosensor protein of various embodiments includes a sequence of aa 1 to aa 53, aa 54 to aa 68, aa 69 to aa 179, aa 180 to aa 437, aa 438 to aa 495, aa 496 to aa 501, aa 502 to aa 765, or combinations thereof of SEQ ID NO: 6. As shown in FIG. 6, the above sequences are for different elements of the biosensor protein of various embodiments.

In various embodiments, the biosensor protein of various embodiments includes a sequence of aa 1 to aa 53, aa 54 to aa 68, aa 69 to aa 179, aa 180 to aa 430, aa 431 to aa 488, aa 489 to aa 494, aa 495 to aa 758, or combinations thereof of SEQ ID NO: 8. As shown in FIG. 8, the above sequences are for different elements of the biosensor protein of various embodiments.

In various embodiments, the biosensor proteins are synthesized from a cell-free reaction medium comprising the expression cassette, the cell extract without viable cells, and the biological or environmental sample or a test compound. The reaction medium of various embodiments can further include phosphoenolpyruvate, nicotinamide adenine dinucleotide, coenzyme A, or an oxolate. The phosphoenolpyruvate, nicotinamide adenine dinucleotide, coenzyme A, or an oxolate relate to a PANOx system for improving synthesis of the biosensor protein of various embodiments. For example, phosphoenolpyruvate (PEP) and nicotinamide adenine dinucleotide (NAD) enables regeneration of ATP and GTP to power the transcription-translation reactions. Oxolate has been shown to improve synthesis as well. The following references explaining the providing PANOx system are incorporated by reference in their entirety: Kim, Dong-Myung, and James R. Swartz. “Regeneration of adenosine triphosphate from glycolytic intermediates for cell-free protein synthesis.” Biotechnology and bioengineering 74.4 (2001): 309-316; Jewett, Michael C., and James R. Swartz. “Rapid Expression and Purification of 100 nmol Quantities of Active Protein Using Cell-Free Protein Synthesis.” Biotechnology progress 20.1 (2004): 102-109.

The following references, which disclose various cell-free methods of protein synthesis, are incorporated herein by reference: U.S. Pat. Nos. 6,548,276; 6,994,986; 7,312,049; 7,312,049; 7,338,789; 7,871,794; 8,298,759; 8,357,529; 9,410,170; U.S. Patent Application Publication No. 2010/0063258; and PCT Patent Application Publication No. WO2016/112258.

In various embodiments, the cell-free systems and methods of detecting bioactive ligands in a biological or environmental sample can further include the step of acquiring the biological or environmental sample. The biological or environmental sample of various embodiments can include samples from a mammalian subject such as a human. Samples from mammalian subjects of various embodiments can include whole blood, whole blood treated with an anticoagulant such as heparin, serum, plasma, plasma treated with an anticoagulant such as heparin urine, feces, sputum, saliva, or sweat. The biological or environmental sample of various embodiments can include water samples; tap water samples; rain/storm water samples; snow samples; waste water samples or water samples from a pond, lake, or river; soil samples; sewage samples; raw sewage samples; post clarifier samples; post biological samples; post filter samples; or effluent samples. In the synthesizing step, the biological or environmental sample of various embodiments can be minimally diluted (e.g. less than 1:1 with a diluent) or undiluted.

In various embodiments, the cell extract without viable cells has components of RNA and polypeptide synthesis machinery capable of expressing the biosensor protein from the polynucleotide of the expression cassette. The cell extract without viable cells of various embodiments are transcriptionally and translationally active.

In the synthesizing step of various embodiments, the biosensor proteins have a properly folded, biologically active conformation when the receptor portions bind bioactive ligands in the biological or environmental sample prior to the polypeptide synthesis machinery reaching a stop codon of an mRNA encoding for the biosensor protein or an improperly folded, biologically inactive conformation when the receptor portions do not bind bioactive ligands prior to the polypeptide synthesis machinery reaching a stop codon of mRNA encoding for the biosensor protein.

The cell-free systems and methods of various embodiments can further include the step of preparing the cell extract without viable cells. Examples of preparing the cell extract without viable cells can include, but are not limited to, physical disruption such as mechanical disruption, liquid homogenization, sonication, freeze-thaw cycle(s), beadbeating, or use of high temperatures and pressures or non-mechanical methods such as using various enzymes or chemicals for lysis. The cell extract without viable cells of various embodiments can be lyophilized/freeze dried. The cell extract of various embodiments can be without viable cells. The cell extract without viable cells of various embodiments can also be a prokaryotic cell extract prepared from prokaryotic cells or a eukaryotic cell extract prepared from eukaryotic cells. Examples of prokaryotic cells include, but are not limited to, various gram stain negative microorganisms, gram stain positive microorganisms, or Escherichia coli (E. coli) or Staphylococcus simulans. Examples of eukaryotic cells include, but are not limited to, fungal cells such as yeast and various mammalian cells including cells from cell lines including Chinese Hamster Ovary (CHO) cells or 293 Human Embryonic Kidney (HEK) cells.

In various embodiments are disclosed cell-free systems and methods of detecting endocrine disrupting compounds in a biological or environmental sample including the step of: providing an expression cassette including a polynucleotide encoding for a biosensor protein comprising: inactive amino- and carboxy-terminal intein splicing domains, a ligand-binding domain capable of binding to endocrine disrupting compounds, wherein the ligand-binding domain is linked to the inactive amino-terminal intein splicing domain on one end and to the inactive carboxy-terminal intein splicing domain on its other end, and a reporter protein activably linked to the ligand-binding domain by at least one of the inactive intein terminal splicing domains; synthesizing biosensor proteins in the presence of a biological or environmental sample and from the expression cassette with a cell extract without viable cells, wherein the biosensor proteins have a properly folded, biologically active conformation when the ligand binding domains bind endocrine disrupting compounds in the biological or environmental sample during synthesis; and detecting the presence of endocrine disrupting compounds in the biological or environmental sample, wherein the reporter proteins of the biosensor proteins having the properly folded, biologically active conformation are capable of generating signals for detection. The ligand binding domain of various embodiment is a ligand-binding domain of a nuclear hormone receptor such as a Type I nuclear receptor/steroid receptor or Type II nonsteroid nuclear receptor. Examples of Type I nuclear receptor/steroid receptors include, but are not limited to, estrogen receptors, androgen receptors, progesterone receptors, mineralcorticoid receptors, or glucocorticoid receptors. Examples of Type II nonsteroid nuclear receptors include, but are not limited to, thyroid hormone receptors, retinoic acid receptors, vitamin D receptors, or peroxisome proliferator-activated receptors. The ligand binding domain or nuclear hormone receptor of various embodiments can be derived from various sources including, for example, mammals such as humans.

In various embodiments are disclosed kits including an expression cassette including a polynucleotide encoding for a biosensor protein and a cell extract that is capable of being combined with at least the expression cassette and a sample or test compound in a cell-free reaction medium for expressing biosensor proteins. The kits of various embodiments can be used for detecting compounds in a sample or for screening compounds for pharmaceutical applications.

In various embodiments are disclosed kits for detecting bioactive ligands in a biological or environmental sample including: an expression cassette including a polynucleotide encoding for a biosensor protein comprising: inactive amino- and carboxy-terminal intein splicing domains, at least a portion of a receptor capable of binding to the bioactive ligand to form a receptor-ligand complex, wherein the at least a portion of a receptor is linked to the inactive amino-terminal intein splicing domain on one end and to the inactive carboxy-terminal intein splicing domain on its other end, and a reporter protein activably linked to the at least a portion of a receptor by at least one of the inactive intein terminal splicing domains; and a cell extract without viable cells that is capable of being combined with at least the expression cassette and a biological or environmental sample in a cell-free reaction medium for synthesizing biosensor proteins; wherein the biosensor proteins have a properly folded, biologically active conformation when the receptor portions bind bioactive ligands in the biological or environmental sample during synthesis and the reporter proteins of the biosensor proteins having the properly folded, biologically active conformation are capable of generating signals for detection.

In various embodiments are disclosed kits screening compounds for pharmaceutical applications including: an expression cassette including a polynucleotide encoding for a biosensor protein comprising: inactive amino- and carboxy-terminal intein splicing domains, at least a portion of a receptor capable of binding to the bioactive ligand to form a receptor-ligand complex, wherein the at least a portion of a receptor is linked to the inactive amino-terminal intein splicing domain on one end and to the inactive carboxy-terminal intein splicing domain on its other end, and a reporter protein activably linked to the at least a portion of a receptor by at least one of the inactive intein terminal splicing domains; and a cell extract without viable cells that is capable of being combined with at least the expression cassette and a biological or environmental sample in a cell-free reaction medium for synthesizing biosensor proteins; wherein the biosensor proteins have a properly folded, biologically active conformation when the receptor portions bind bioactive ligands in the biological or environmental sample during synthesis and the reporter proteins of the biosensor proteins having the properly folded, biologically active conformation are capable of generating signals for detection.

The kit of various embodiments can include effective concentrations of phosphoenolpyruvate, nicotinamide adenine dinucleotide, coenzyme A, an oxolate. The kit of various embodiments also can include substrates for the enzymatic reporter protein to generate visual signals or a control concentration of the bioactive ligand.

The following examples illustrate the various embodiments of the present invention. Those skilled in the art will recognize many variations that are within the spirit of the present invention and scope of the claims.

In various embodiment are disclosed a Rapid Adaptable Portable In-vitro Detection biosensor platform (RAPID) for detecting ligands that interact with nuclear hormone receptors (NHRs). The RAPID platform can be adapted for field use, allowing rapid evaluation of endocrine disrupting chemicals (EDC) presence or absence in environmental samples, and could also be applied for drug screening. The biosensor is based on an engineered, allosterically-activated fusion protein, which contains the ligand binding domain from a target NHR (human thyroid receptor b in this work). In vitro expression of this protein using cell-free protein synthesis (CFPS) technology in the presence of an EDC leads to activation of a reporter enzyme, reported through a straightforward colorimetric assay output. In this work, we demonstrate the potential of this biosensor platform to be used in a portable “just-add-sample” format for near real-time detection. We also demonstrate the robust nature of the cell-free protein synthesis component in the presence of a variety of environmental and human samples, including sewage, blood, and urine. The presented RAPID biosensor platform is significantly faster and less labor intensive than commonly available technologies, making it a promising tool for detecting environmental EDC contamination and screening potential NHR-targeted pharmaceuticals.

Biosensors can be life-changing devices, with uses ranging from daily glucose monitoring for diabetes patients to the rapid detection of toxins in the environment^{48,49}. When biosensors provide the required degree of specificity and sensitivity in combination with more rapid assay times, they are excellent alternatives to traditional detection methods⁵⁰. Biosensing systems are available in various formats, from cell based systems with complex metabolic pathways to less complex in vitro systems. Cell-based systems can have a broader spectrum of detection capabilities, however, they are hindered by transmembrane transport limitations, the need to maintain cell viability and stability, time-consuming preparation, and protracted assay times^{51,52}. In contrast, in vitro methods are commonly faster, more straightforward, simpler to store, and less expensive. Here we present a versatile, near-real time in vitro biosensor for detecting ligands that bind nuclear hormone receptors (NHRs)

In vitro expression of this protein using cell-free protein synthesis (CFPS) technology in the presence of an EDC leads to activation of a reporter enzyme, reported through a straightforward colorimetric assay output. In this work, we demonstrate the potential of this biosensor platform to be used in a portable “just-add-sample” format for near real-time detection. We also demonstrate the robust nature of the cell-free protein synthesis component in the presence of a variety of environmental and human samples, including sewage, blood, and urine. The presented RAPID biosensor platform is significantly faster and less labor intensive than commonly available technologies, making it a promising tool for detecting environmental EDC contamination and screening potential NHR-targeted pharmaceuticals.

In this work, we introduce the Rapid Adaptable Portable Invitro Detection biosensor (RAPID). This assay system combines our existing multi-domain biosensor protein design with rapid and efficient CFPS technology to overcome specific limitations of both in vitro and cell-based assays. In this system, the biosensor fusion protein is expressed using a CFPS system in the presence or absence of an unknown EDC sample. An engineered reporter enzyme domain on the biosensor protein exhibits ligand-dependent activity, resulting in a simple, colorimetric readout. Unique CFPS characteristics, including its chemically accessible reaction environment, robustness, scalability, and control^{53,54,55}, make this technology a powerful biosensing platform for both simple and complex detection applications. In addition, the ability to lyophilize the CFPS components enables this type of biosensor to be stockpiled for emergencies and biothreat situations. Further, the robustness of the sensor design and simplicity of its visual readout could facilitate field-deployment, where assays of environmental samples could be carried out by minimally trained personnel in the absence of any conventional laboratory equipment. By leveraging the advantageous traits of CFPS, we have generated a highly practical and effective CFPS biosensor for uses in detecting toxic EDCs, as well as potentially valuable therapeutics against this important drug target class.

### Experimental Section

### Materials.

The ligands used for this paper, 3,3′,5-triiodothyroacetic acid (TRIAC, 95%), 17-β-estradiol (E2), and 3,3′,5-triiodo-L-thyronine sodium salt hydrate (T3, 95%), were purchased from Sigma-Aldrich.

### Biosensor Design and Construction.

The pET-based plasmid encoding the biosensor protein (MBP-IN-hTRβ-IC-βlac as illustrated in FIG. 9) is based on our previously reported biosensor design for thyroid receptor (TR) ligands⁵⁶. FIG. 9 shows the protein construct for the RAPID biosensor. It includes an MBP at its N-terminus, a mini-intein splicing domain with an inserted NHR ligand binding domain (from hTRβ in this work), and a C-terminal reporter enzyme (β-lac in this work). The presence of ligand during expression of the protein changes the structure of the biosensor and improves accessibility of the reporter enzyme. FIG. 11A shows alternative embodiments, where the polypeptide construct and biosensor protein includes a CBDP at its N-terminus, a mini-intein splicing domain with an inserted a receptor portion such as an NHR ligand binding domain, and a C-terminal reporter enzyme. FIG. 11B shows other alternative embodiments, where the polypeptide construct and biosensor protein includes a reporter enzyme at its N-terminus, a mini-intein splicing domain with an inserted a receptor portion such as an NHR ligand binding domain, and a C-terminal CBDP. The biosensor fusion protein was inserted into the DHFR control plasmid supplied with the PureExpress® In Vitro Protein Synthesis Kit (New England Biolabs), which includes a T7 promoter to regulate expression of the target protein. Construction of the biosensor gene was accomplished by stepwise insertion of DNA segments encoding the maltose-binding domain (MBP), the intein human TR fusion (IN-hTRβ-IC), and the β-lactamase reporter protein (β-lac), where the resulting biosensor fusion gene replaces the DFHR expression control gene. In this case, the MBP was taken from the commercially available pMal-c2 expression vector (New England Biolabs), the IN-hTRβ-IC segment was taken from our previously reported TR biosensor plasmid⁵⁶, and the β-lac reporter protein was taken from a previously reported intein fusion expression plasmid⁵⁷.

### Cell Extract Preparation.

Cell extract preparation was performed as previously described⁵⁸. Briefly, 5 ml of LB media was inoculated using E. coli BL21.DE3* strain in a cell culture tube. The culture was incubated overnight at 37° C. while shaking at 280 rpm. The culture was transferred to 100 ml LB media and upon reaching OD 2.0, it was transferred to 1 liter LB media in Tunair flask. T7 RNA polymerase was overexpressed by inducing the culture with 1 mM Isopropyl 2-D-1-thiogalactopyranoside (IPTG) at OD 0.6. The cells were harvested at the end of the exponential phase by centrifugation at 6000 RCF for 10 min at 4° C. The cells were washed by suspending in pre-chilled Buffer A (10 mM Tris-acetate pH 8.2, 14 mM magnesium acetate, 60 mM potassium glutamate, and 1 mM dithiothreitol (DTT)), and subsequently centrifuged at 6000 RCF for 10 min 4° C. The cells were resuspended in 1 ml Buffer A per gram cells and homogenized using EmulsiFlex French Press homogenizer at 20000 psi. The lysate was clarified by centrifugation at 12000 RCF for 30 min at 4° C. The supernatant was incubated at 37° C. for 30 min while shaking at 280 rpm, flash frozen in liquid nitrogen, and then stored at −80° C. for later use as cell extract for CFPS.

### Lyophoilizing Biosenser System.

For lyophilized biosensor systems, CFPS reagents were mixed and lyophilized as described previously^{59,60} with slight modifications including that all reagents necessary for CFPS were combined and lyophilized together. Briefly, CFPS components were added to a prechilled tube in the following order while the tube rested on the ice: deionized water, magnesium glutamate, PANOxSP, and lastly the plasmid. The reaction mixture was mixed gently and transferred to 1.5 ml Eppendorf tubes in 250 μl aliquots. Tubes was quickly placed in liquid nitrogen container to flash freeze the reaction. The samples were lyophilized using FreeZone 2.5 Liter Benchtop Freeze Dry System (LABCONCO, Kansas City, Mo.) with the operation condition of −50° C. and <120 mTorr for 8 hr.

### Cell-Free Protein Synthesis Reaction.

The CFPS reactions were performed in 96 well plate using PANOxSP system for 20 to 180 min at 37° C.⁶¹. The reactions contained 25 volume percent cell extract, 1.20 nM plasmid and following components all from Sigma-Aldrich (St. Louis, Mo.): 10 to 15 mM magnesium glutamate, which was optimized based on the extract, 1 mM 1,4-Diaminobutane, 1.5 mM Spermidine, 33.33 mM phosphoenolpyruvate (PEP), 10 mM ammonium glutamate, 175 mM potassium glutamate, 2.7 mM potassium oxalate, 0.33 mM nicotinamide adenine dinucleotide (NAD), 0.27 mM coenzyme A (CoA), 1.2 mM ATP, 0.86 mM CTP, 0.86 mM GTP, 0.86 mM UTP, 0.17 mM folinic acid, 2 mM of all the canonical amino acids except glutamic acid. For experiments requiring measurement of protein production yield using a scintillation counter, 5 mM 1-[U-14C] Leucine (PerkinElmer, Waltham, Mass.) was added to the reaction, and protein yield was calculated based on total and washed counts described previously⁶².

### Environmental and Human Samples Tested in Cell-Free Protein Synthesis.

Tap water, storm water, and pond water were collected at various locations in Utah County, USA. Soil and snow samples were collected in Salt Lake County, Utah, where soil samples were extracted into ddH2O at a one to one (weight to volume) ratio. All of the wastewater treatment samples were collected from the Provo city water reclamation facility. Raw sewage was influent of the plant. Post clarifier sample was after primary sedimentation basins. Post biological sample was the effluent of aeration basins with activated sludge. Post filter sample was the activated sludge process effluent (final clarifier effluent) passed through anthracite filters. The effluent sample was the final product of the plant after chlorination and dechlorination treatments. Single donor human whole blood-Na Heparin sample was obtained from Innovative Research (Peary Ct, Novi, Mich.). Urine samples were obtained from volunteers.

### Hormone Biosensor Assay.

The Hormone biosensor assay was performed in 2 stages. Stage 1: CFPS of the biosensor protein in 96 well plate for 20 min in the presence of 0 to 10 mM TRIAC, T3, or E2 dissolved in Dimethyl sulfoxide (DMSO). For consistency all CFPS reactions were adjusted to have 5 volume percent DMSO. Stage 2: After 20 min, the reactions were diluted 104-fold into PBS buffer, of which 25 ml was transferred into each well of a UV-transparent Corning® 96 well plate. To each well, 175 ml of 228.6 mM nitrocefin in PBS was additionally added to the wells at the same time to achieve a final nitrocefin concentration of 200 mM. The plates were then directly quantified via plate reader (BioTek Synergy Mx) for a nitrocefin-based beta-lactamase activity assay⁶³. Specifically, the absorbance was read at 390 and 490 nm wavelengths for unreacted and reacted substrate nitrocefin, respectively. Measurements were repeated at 1 min intervals, with 10 sec shaking at each interval to mix, for 15 min. At the end of the assay, the absorbance was read at 760 nm to provide a relative background level for the assay. The rate of nitrocefin conversion was determined at each ligand concentration using the time course measurements, and the resulting rates were used to determine the half maximal effective ligand concentration (EC₅₀).

### Analysis of Hormone Biosensor Assay Results.

The nitrocefin conversion value (NCV) was calculated using Equation 1. The A390 is lₘₐₓ of the yellow substrate nitrocefin, while A490 is the lₘₐₓ of the red nitrocefin conversion product, and A760 is background absorbance of each well. In order to maximize the signal-to-noise ratio, the time point with the maximum difference between the NCVs of the negative control (zero ligand) and maximum ligand concentration was selected to calculate the dose-response curves. The Four-Parameter Logistic Function (Equation 2) was fitted to this data to yield the half maximal effective concentration (EC₅₀)⁶⁴. Parameters “a” and “b” define lower and upper plateau value of the function, respectively, while “k” is the slope factor.

$$\begin{matrix}Nitrocefin Conversion Value ( N V C ) = \frac{{A}_{490} - {A}_{760 ( median of all reaction wells )}}{{A}_{390} - {A}_{760 ( median of all reaction wells )}} & ( 1 ) \\ Predicted N V C = a + \frac{b - a}{1 + ( exp ( k ( log ( ligand concentration ) - log ( {EC}_{50} ) ) )} & ( 2 )\end{matrix}$$

To generate percentage dose-response graphs, values and predicted values from the fitted function were normalized based on the equation 3.

$$Normalized Dose Response = \frac{( N C V ) - Min ( N C V )}{Max ( N C V ) - Min ( N C V )} * 100 \% \qquad ( 3 )$$

The overall quality of the assays was assessed using Z′ factor, signal-to-noise ratio (S/N), and signal to background ratio (S/B) parameters. The parameters were calculated using a previously described method^{65,66}. Also, the limit of detection (LOD) was calculated based on IUPAC methodology by finding the corresponding concentration value for blank measurement added to its three times standard deviation⁶⁷.

Scheme of the RAPID Biosensor to Detect Chemicals that Target NHRs.

The biosensor assay includes two steps: 1—CFPS reaction to produce the biosensor, 2—colorimetric assay to quantify the biosensor protein activation. The presence of ligand during protein synthesis activates the biosensor by altering the conformation of biosensor enzyme and increasing the nitrocefin assay signal.

### Result and Discussion

### RAPID Biosensor Design and Rationale.

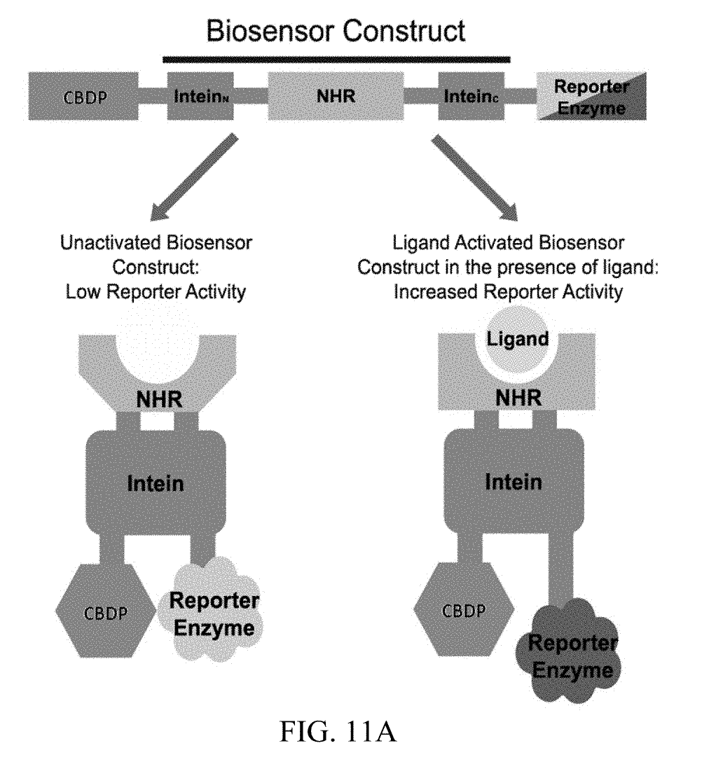

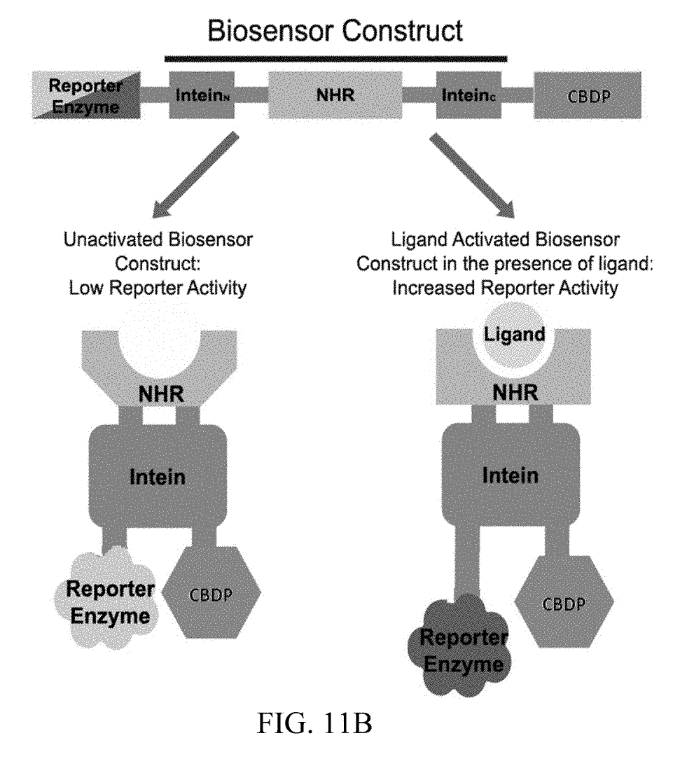

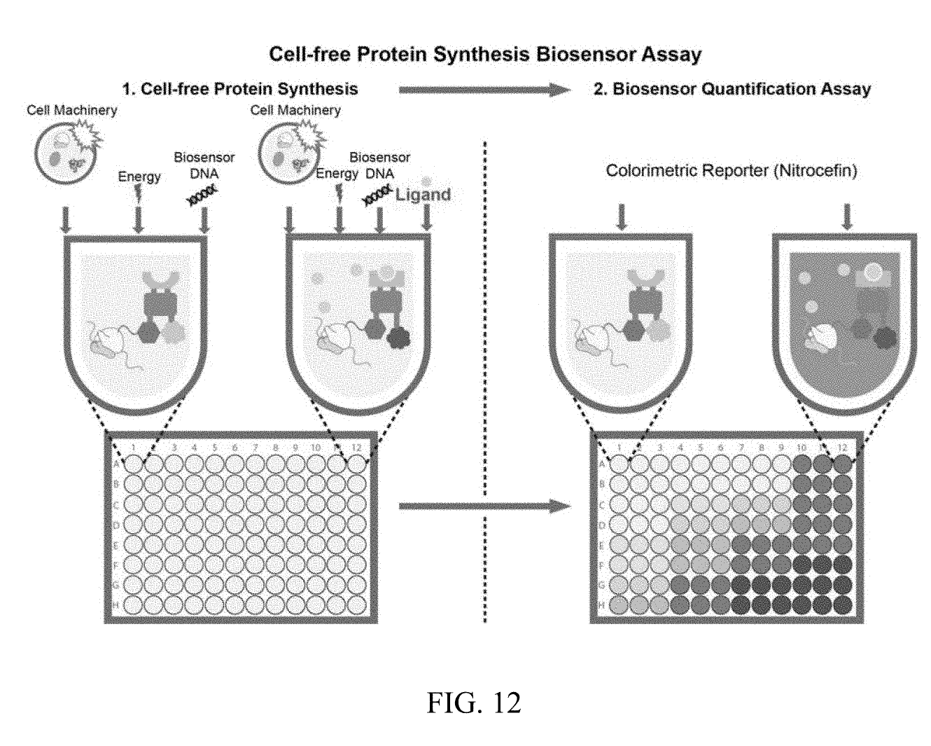

Here we report the RAPID (Rapid Adaptable Portable In-vitro Detection) biosensor for NHR-binding ligands. The goal of this work was to create a near real-time biosensor platform by combining our previous cell-based allosterically activated, fusion protein approach⁵⁶ with the open flexibility of CFPS systems⁶⁸. The fusion protein consists of four domains including: 1) maltose binding domain, which improves the solubility of the fusion protein⁴³; 2) mini-intein domain, which acts as a stabilizing domain for the NHR domain⁴³; 3) NHR ligand binding domain, which is the heart of biosensor and acts as a switch to activate the reporter enzyme; and 4) the reporter enzyme. An interaction between a ligand/chemical and the NHR ligand binding domain causes a conformation change which results in improved reporter protein activity as preciously described⁶⁹. Hence, a signal results from the presence of a chemical/ligand that binds the NHR ligand binding domain during protein synthesis (FIGS. 9, 12). FIG. 12 shows a Scheme of the RAPID biosensor to detect chemicals that target NHRs. The biosensor assay includes two steps: 1-CFPS reaction to produce the biosensor, 2-colorimetric assay to quantify the biosensor protein activation. The presence of ligand during protein synthesis activates the biosensor by altering the conformation of biosensor enzyme and increasing the nitrocefin assay signal.

Due to the cell-free nature of CFPS, there is no membrane transfer limitation for chemicals that might target NHRs⁷⁰, while the direct translation of the sensor protein provides a fast, inexpensive, and convenient assay for the presence of EDC activity in unknown test chemicals.

The initial step in creating the CFPS-based RAPID biosensor was to re-engineer the reporter protein domain for a rapid and straightforward colorimetric assay readout. Our previous bacterial biosensor platform employed the thymidylate synthase reporter enzyme to enable growth phenotype changes⁴³. Unfortunately, in vitro assays for thymidylate synthase activity are cumbersome and require oxygen-sensitive reagents. For these reasons, the β-lactamase (β-lac) enzyme was selected to replace the thymidylate synthase enzyme due to its similarity in size and commercially available colorimetric activity assay.

To characterize our RAPID biosensor, the human thyroid receptor β (hTRβ) was chosen for the initial ligand binding domain due to its robust behavior in our bacterial biosensor⁵⁶. It also has high sensitivity and selectivity to TRIAC, a potent agonist, with a half-maximal effective concentration value (EC₅₀) reported at 70 nM. Cloning work to incorporate the β-lac reporter and hTRβ ligand binding domain into the fusion protein is described in the methods section, with the final fusion protein sequence illustrated in FIG. 9.

### Cell-Free Protein Synthesis of the Reporter Fusion Protein.

The resulting fusion protein, containing the hTRβ ligand binding domain and β-lac, was expressed in an E. coli-based CFPS system as detailed in the methods section. To elucidate the mechanism of activation, total protein titer and protein solubility were measured by tracking the incorporation of C-14 radiolabeled leucine (FIG. 10). FIG. 10 shows the cell-free protein synthesis of the biosensor fusion protein with protein production yields reported for increasing reaction times and in the presence of three levels of the ligand T3 (total protein=dark bars, soluble protein=light bars, reaction volume was 20 ml). The 92 kD MBP-IN-hTRβ-IC-βlac fusion protein was expressed at yields up to 700 mg/mL in 3 hr and the expression level was unaffected by the presence of T3 ligand (FIG. 10). Also, the protein solubility yields were consistently greater than 85% (FIG. 10).

### Hormone Biosensor Assay.

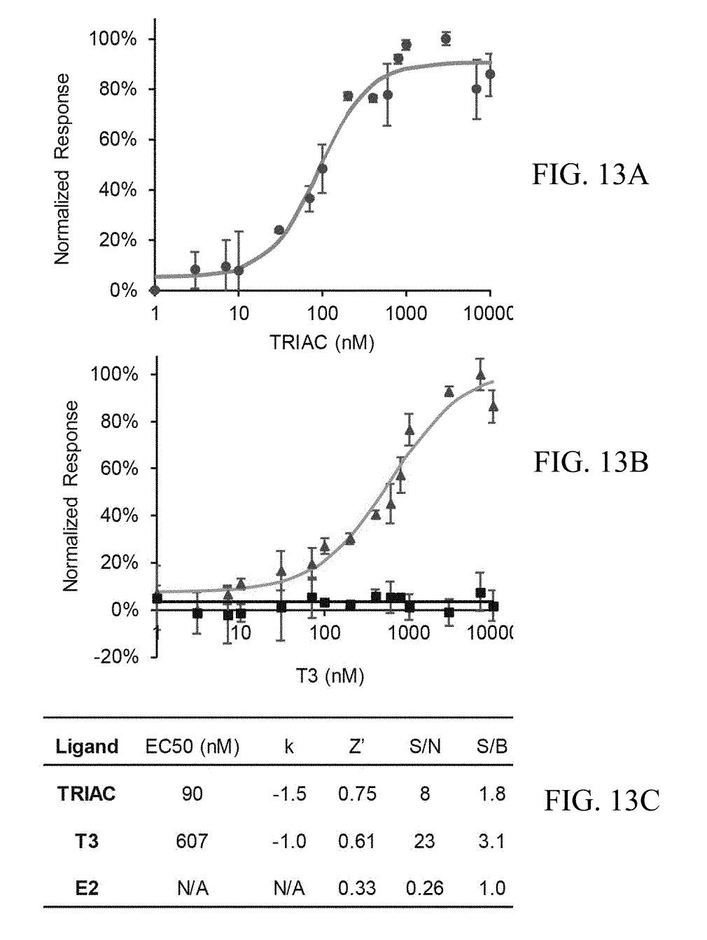

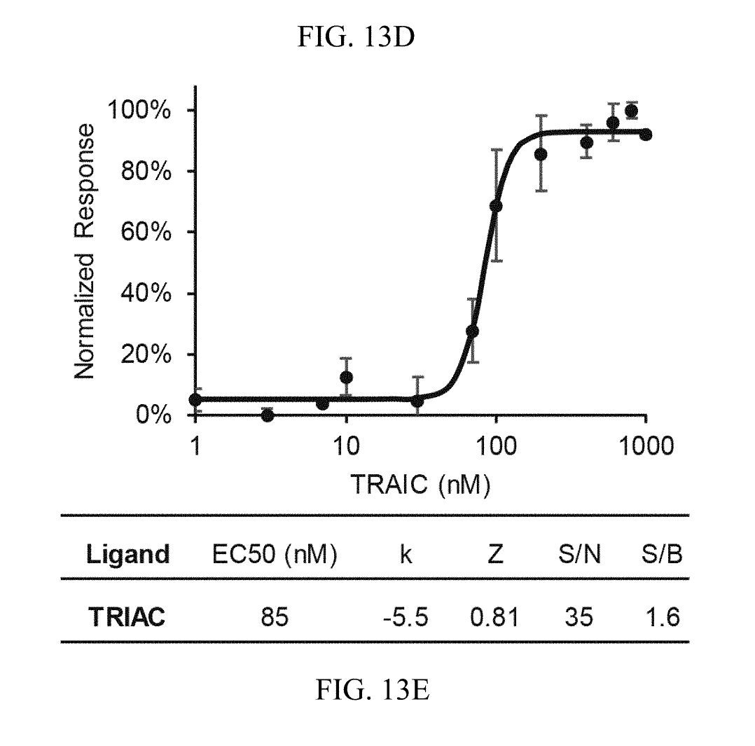

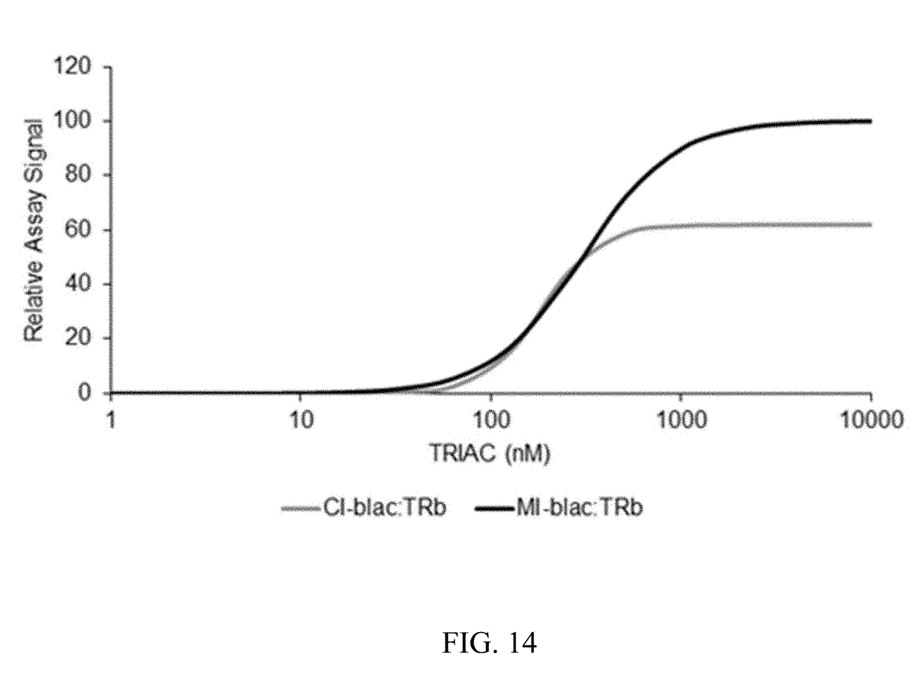

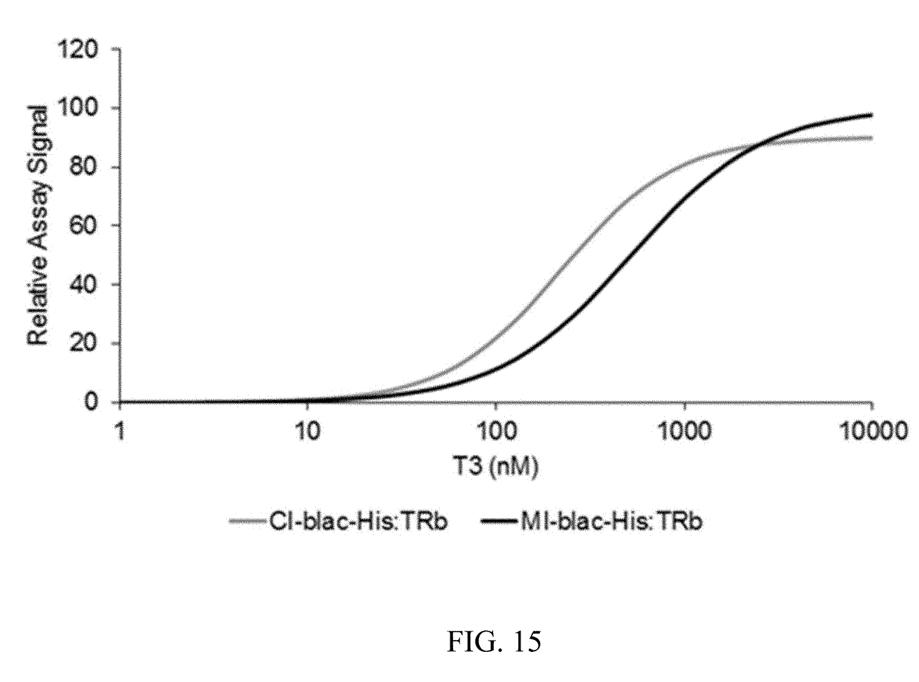

The hormone biosensor assay consists of two steps as illustrated in FIG. 12. First, cell-free expression of the MBP-IN-hTRβ-IC-β-lac reporter fusion protein is performed in the presence of the sample to be tested. The resulting protein is then subjected to a colorimetric reporter enzyme activity assay, where NHR-ligand binding is reflected in the activity of the reporter enzyme domain (β-lac). The hormone sensing capability of this assay was assessed with 3 known endocrine disrupting chemicals; two chemicals that are known to target hTRβ (TRIAC and T3), and a negative control (estrogen) that targets the human estrogen receptor NHR but does not target hTRβ. The results are reported in FIG. 13A-C, where the EC₅₀, Z′ factor, signal-to-noise ratio (S/N), and signal to background ratio (S/B) are calculated for each chemical. FIG. 13A shows the dose-response curve for the hTRβ biosensor in the presence of TRIAC. FIG. 13B shows the dose-response curves of the hTRβ biosensor in the presence of T3 (triangles), and E2 (squares). FIG. 13C shows the half-maximal effective concentration (EC₅₀), slope factor (k), Z′ factor, signal to noise ratio (S/N), and signal to background ratio (S/B) for the responses against TRIAC, T3, and E2. The solid lines represent fitted nitrocefin conversion values, the markers represent the average measured values, and the error bars represent one standard deviation for n=2. The Z′ factor was between 0.5 to 1 for all assays, indicating “an excellent assay” for screening and sensing^{65,66}. The measured EC₅₀ for TRIAC and T3 were 90 and 607 nM, respectively, which correspond well to the EC₅₀ from our previous studies with the bacterial biosensor, 70 and 580 nM respectively (FIGS. 13A-C)⁵⁶. Also, the calculated LOD were 48 and 75 nM, respectively for TRIAC and T3. As expected, a statistically significant signal was not observed with the estrogen negative control (FIG. 13B, Square markers, p-value of 0.84). TRIAC was 7-fold more potent than T3 against TRβ which is similar to our bacterial biosensor at 8-fold and other reported sensors at 6-fold⁷¹. Although some in vitro binding and transactivation assays can detect ligands with higher sensitivity, the simplicity, speed, and the lack of toxicity or cell-uptake complications make the RAPID system a strong candidate for screening of NHR binding ligands^{71,72,73}. FIG. 14 shows example dose responses of hTRβ biosensor proteins of various embodiments with MBP as shown in FIG. 9 (Black) and with CBDP as shown in FIG. 10 (Gray) the presence of TRIAC. FIG. 15 shows example dose responses of hTRβ biosensor proteins of various embodiments with a polyhistidine-tag and including MBP as shown in FIG. 9 (Black) or CBDP as shown in FIG. 10 (Gray) the presence of T3 hormones.

One considerable strength of our RAPID biosensor is speed of the assay, with the total time needed to generate clear results being less than 30 min. Alternatively, mammalian-based assays may take days to weeks to complete and bacterial-based assays take 24-36 hours^{56,74}. Another strength of the cell-free system is the elimination of confounding issues associated with membrane transport of test chemicals, unknown or unintended side effects related to cell growth or toxicity, or cross activation of NHRs⁶⁸. In contrast to other in vitro techniques, a further advantage of our system is that there is no need for any purification or complex enzyme stabilization steps 75. Furthermore, the modular nature of the fusion protein opens the possibility of optimizing the system by rapidly incorporating new reporter enzymes, while also expanding the RAPID biosensor to include diverse nuclear hormone receptors for human and animal applications⁷⁶.

### Lyophilized Biosensor.

To develop our RAPID biosensor platform for potential field use (i.e. outside of the laboratory), we assessed the possibility of lyophilizing the CFPS biosensor components. Previously, we reported lyophilized cell extracts remained active after 90 days of storage at room temperature, and demonstrated the potential for CFPS to be used in biotherapeutic protein production⁷⁷. For this work, all essential elements, including cell extract, plasmid encoding the fusion protein, and necessary small molecule additives were combined and lyophilized at the same time, to create a “just-add-sample” CFPS assay. The results illustrate that lyophilized CFPS performed similarly to freshly prepared CFPS in detecting TRIAC (85 nM EC₅₀, −5.5 k, 0.81 Z′, 35 S/N, 1.6 S/B, 59 nM LOD) (FIGS. 13D and 13C). FIGS. 13D and 13C are dose-response graph and statistical analysis results for the RAPID biosensor with lyophilized CFPS components in the presence of TRIAC. The solid line represents fitted nitrocefin conversion values, while circle markers represent the measured values. Error bars represent one standard deviation for n=2. Thus, the RAPID biosensor has the potential to be used as a field assay for in situ real-time detection of EDCs in essential infrastructure, such as watersheds.

### CFPS Performance in Different Environmental Samples.

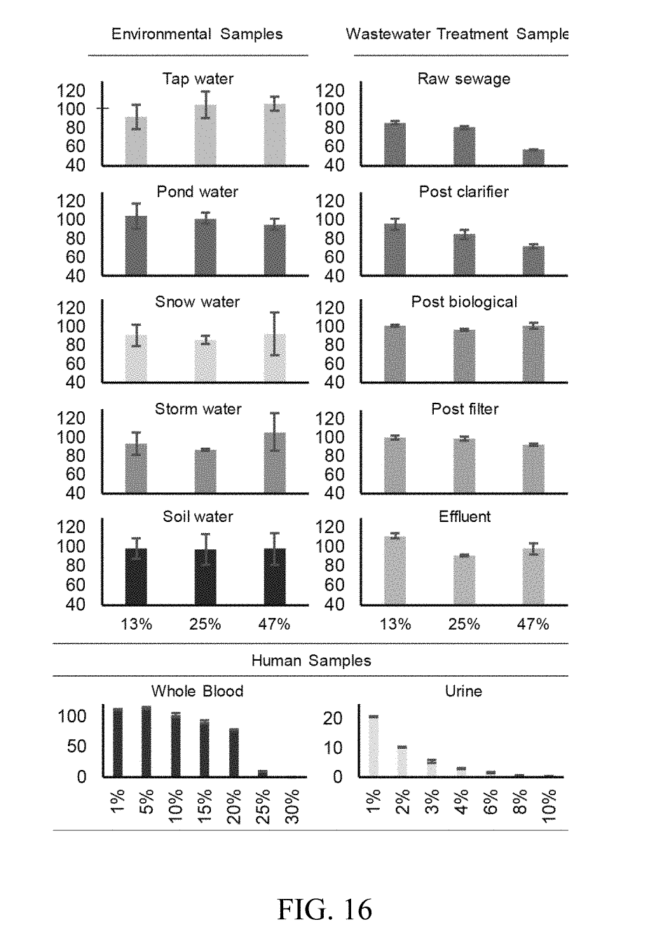

To understand the utility of this new NHR biosensor for evaluating environmental samples, we tested the performance of the CFPS system—a sensitive component of the RAPID biosensor—in various untreated water sources, raw sewage, and human bodily fluids (FIG. 16). FIG. 16 shows the protein production capability of CFPS in the presence of environmental and human samples. In all cases model protein GFP is expressed and the production level (y-axis) is normalized to GFP production in a standard CFPS with 100 corresponding to 100% of the GFP production level in standard CFPS. Each sample type is described in the methods section and the x-axis corresponds to the final concentration for the sample in the CFPS reaction by volume percent. The error bars represent one standard deviation for n=3. For all of the samples, CFPS produced a model protein GFP at sufficient protein production levels necessary for the biosensor assay. The water samples (tap, pond, snow, storm) and samples from various stages of a wastewater treatment plant did not significantly effect CFPS levels, with the exceptions being raw sewage wastewater and post clarifier wastewater. However, even after adding 47% by volume raw sewage or post clarifier wastewater to CFPS reactions, greater than 50% of protein production level was maintained. The robustness of CFPS across diverse environmental samples indicates the potential for use in diverse environmental monitoring situations.

Beyond environmental and wastewater samples, we examined CFPS tolerance to human medical samples, including blood and urine. Greater than 60% of the original CFPS activity was retained in reactions containing up to 20% by volume human blood. Additionally, we note that the blood we used in this work contained heparin as anticoagulant in lieu of EDTA, because EDTA at high concentrations can sequester magnesium and inactivate CFPS⁶¹. Expectedly, human urine, which contains a significant concentration (˜280 mM) of the protein denaturant urea⁷⁸, had the greatest impact on CFPS activity. However, CFPS activity remained detectable at up to 8% by volume urine (1% original activity, with a standard deviation of 0.05%). To account for significant yield changes cause by urine samples, a control CFPS reaction with a model protein such as GFP could be used in combination with the biosensor to ensure consistent dilution of the CFPS biosensing protein in the second colorimetric stage of the biosensor assay. Overall, the ability of CFPS to tolerate high levels of various contaminants, such as organic matter, bacteria, blood, urine and wastewater demonstrates its robustness as a biosensing platform.

### NHR RAPID Biosensor Performance in an Environmental Sample.

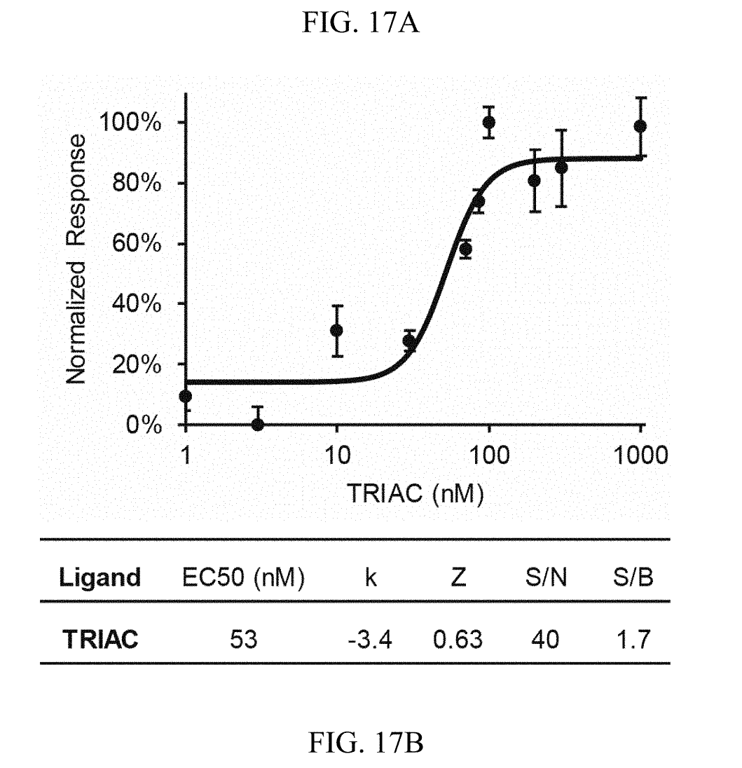

Raw sewage was chosen to investigate how the composite biosensor was affected by the presence of an actual environmental sample. CFPS reactions containing 40% final volume raw sewage and TRIAC at varying concentrations were reacted for 20 mins. Subsequently, the reactions were diluted and assayed using the described colorimetric assay. The resulting RAPID biosensor maintained its sensitivity for TRIAC (53 nM EC₅₀, −3.4 k, 0.63 Z′, 40 S/N, 1.7 S/B, 28 nM LOD) (FIGS. 17A and 17B). FIGS. 17A and 17B show dose-response graph and statistical analysis results for the RAPID biosensor in the presence of TRIAC and 40% by volume raw sewage. The solid line represents fitted nitrocefin conversion values, the circle markers represent the measured values, and the error bars represent one standard deviation for n=2.

### Conclusion

Here we have developed a new RAPID biosensor platform for chemicals that target nuclear hormone receptors using a quick, versatile cell-free protein synthesis approach. The developed biosensor has some key advantages over existing biosensors, including near real-time readout, the potential for portable field use, and reduced labor and cost requirements. This biosensor is also a promising tool for studying various NHR-binding ligands in a high-throughput manner. Additionally, the ability of CFPS to perform protein synthesis in different human and environmental samples, showed strong potential of the biosensor for detecting NHR-targeting compounds directly, without requiring purification or modification of the sample. Overall the RAPID biosensor is an attractive alternative to currently available technology and provides a fast, versatile platform for detecting potential NHR-binding ligands including EDCs and therapeutics.

The following examples illustrate the various embodiments of the present invention. Those skilled in the art will recognize many variations that are within the spirit of the present invention and scope of the claims.

In various embodiments are disclosed a Rapid Adaptable Portable In-vitro Detection biosensor platform (RAPID) for detecting chemicals that interact with the human estrogen receptor β (hERβ). This biosensor consists of an allosteric fusion protein, which is expressed using cell-free protein synthesis technology and is directly assayed by a colorimetric response. The resultant biosensor successfully detected known EDCs of hERβ including BPA, E2, and DPN at similar or better detection range than an analogous cell-based biosensor, but in a fraction of time. We also engineered cell-free protein synthesis reactions with RNAse inhibitors to increase production yields in the presence of human blood and urine. The RAPID biosensor successfully detects EDCs in these human samples in the presence of RNAse inhibitors. Engineered cell-free protein synthesis facilitates the use of protein biosensors in complex sample matrices without cumbersome protein purification.

As disclosed in prior embodiments, we introduced our Rapid Adaptable Portable In-vitro Detection biosensor platform (RAPID) for determining EDC activity⁷⁹. This biosensor platform relies on exploiting the basic cellular mechanism of EDC activity in the following manner. An allosterically activated fusion protein containing the ligand-binding domain of a nuclear hormone receptor (NHR) and the reporter enzyme β-lactamase is synthesized in a cell-free protein synthesis (CFPS) reaction in the presence of a sample. If the sample contains NHR binding ligands, folding of the sensor protein is stabilized and an increase in colorimetric signal is generated real-time. Unlike many emerging biosensor technologies, the RAPID biosensor is not analyte specific. Instead, the sensor detects binding interactions with a given NHR.

Our previous work demonstrated the utility of this biosensor to detect and screen EDCs that interact with the Human thyroid receptor β (hTRβ). In this work, we demonstrate the modular nature of the RAPID biosensor by replacing the hTRβ domain with that of the human estrogen receptor β (hERβ) to detect XEs. The CFPS reaction allows direct utilization of the biosensor protein without cell culture or protein purification. We further engineer the CFPS biosensing reaction using RNAse inhibitors to achieve significantly enhanced protein synthesis, and thereafter XE detection, in human blood and urine. To our knowledge, this is the first report of biosensor proteins produced in CFPS reactions engineered to overcome adverse sample matrix effects.

### Experimental Section

### Materials.

All EDC ligands were purchased from Sigma-Aldrich (St. Louis, Mo. USA): diarylpropionitrile (DPN), bisphenol A (BPA), β-estradiol (E2), and TRIAC (3,3′,5-triiodothyroacetic acid). Blood samples were purchased from Innovative Research (Novi, Mich., USA). The human donor urine was donated anonymously by the BYU health center, nitrocefin was purchased from Cayman Chemical (Ann Arbor, Mich. USA), and murine RNAse inhibitor was purchased from New England Biolabs (Ipswich, Mass.).

### Biosensor Design and Construction.

The pDB-MI-hERβ-β-lac construct was created in a similar manner as our previous biosensor construct⁷⁹. The pDB vector that encodes for the biosensor protein complex possesses four domains: maltose binding domain (MBD), human estrogen receptor β (hERβ), β-lactamase, and two split intein segments between MBD and hERβ, and hERβ and β-lactamase. The gene encoding this protein is under control of the T7 promoter. The construction of the vector was detailed previously⁷⁹.

### Cell Extract Preparation.

Cell extract was prepared as described previously⁸⁰. Briefly, an inoculum of E. coli BL21* (DE3) strain was grown overnight in 5 mL of LB media at 37° C. and 280 rpm. The culture was added to 100 mL of LB media and grew until OD600 reached 2.0. At 2.0 OD600, the 105 mL culture was transferred to 1 L of LB media in a tunair baffled flask. At 0.6 OD600, isopropyl β-D-1-thiogalactopyranoside (IPTG) was added to achieve a final concentration of 1 mM. Cells were harvested at the end of the exponential growth phase using centrifugation at 6000 RCF for 10 min at 4° C. These were then washed with 4° C. Buffer A (10 mM Tris-acetate pH 8.2, 14 mM magnesium acetate, 60 mM potassium glutamate, and 1 mM dithiothreitol (DTT)) and then collected using an identical centrifugation cycle. Next, the pellet was re-suspended in Buffer A (1 g of cell/mL of buffer A), and homogenized with 3 passes through an EmulsiFlex French Press at 20000 psi. The homogenized cells were centrifuged at 12000 RCF for 30 min at 4° C. to clear the lysate. The supernatant was incubated in a shaking incubator for 30 min at 280 rpm and 37° C. The extract was flash-frozen in liquid nitrogen for 60 seconds before being stored at −80° C.

### Cell-Free Protein Synthesis Reaction.

CFPS reactions took place in a 96-well plate at 37° C. for 2 hours. PANOxSP was used as an energy source (25% reaction volume of the cell extract, 1.2 nM plasmid, 12 to 15 mM magnesium glutamate, 1 mM 1,4-diaminobutane, 1.5 mM Spermidine, 33.3 mM phosphoenolpyruvate, 10 mM ammonium glutamate, 175 mM potassium glutamate, 2.7 mM potassium oxalate, 0.33 mM nicotinamide adenine dinucleotide, 0.27 mM coenzyme A, 1.2 mM ATP, 0.86 mM CTP, 0.86 mM GTP, 0.86 mM UTP, 0.17 mM folinic acid, 2 mM of all canonical amino acids with the exception of glutamic acid⁸¹. Expressed protein was quantified by addition of 5 μM radiolabeled (U-14C) leucine (PerkinElmer, Waltham, Mass.) to the CFPS reaction. Varying concentrations (0 to 200 μM) of ligands were added to the CFPS reaction. In case of human sample testing, 20% and 10% of whole blood and urine (v/v), respectively were added to the CFPS in addition to the EDC ligands.

### RAPID Hormone Biosensor Assay.

The biosensor assay was performed in 2 stages as described previously⁷⁹. Stage 1: CFPS of the biosensor protein in 96 well plate for 2 hours in the presence of 0 to 10 mM DPN, BPA, TRIAC, or E2 dissolved in dimethyl sulfoxide (DMSO). The presence of the hormone receptor ligand is thought to enhance correct folding of the biosensor protein, leading to increased activity upon expression. For consistency, all CFPS reactions were adjusted to have final 5% (v/v) DMSO concentration. Stage 2: After 2 hours, the reactions first were diluted 13-fold into PBS buffer, of which 25 ml was transferred into each well of a UV-transparent Corning® 96 well plate. To each well, 175 ml of 228.6 mM nitrocefin in PBS was additionally added at the same time to achieve a final nitrocefin concentration of 200 mM and overall 104-fold dilution of CFPS to eliminate signal overflow. The plates were then directly quantified via plate reader (BioTek Synergy Mx) for a nitrocefin-based beta-lactamase activity assay. Specifically, the absorbance was read at 390 and 490 nm wavelengths for unreacted and reacted substrate nitrocefin, respectively. Measurements were repeated at 1 min intervals, with 10 sec shaking at each interval to mix, for 40 min. At the end of the assay, the absorbance was read at 760 nm to provide a relative background level for the assay. Nitrocefin conversion was determined for each ligand concentration at each point in time, and the data from the time point with the largest difference in nitrocefin conversion was fit to a four-parameter logistic function to obtain the effective ligand concentration (EC₅₀)⁷⁹.

The quality of the assays was assessed using the Z′factor, signal-to-noise ratio (S/N), and signal to background ratio (S/B), and these were calculated using previously described methods_{82,83}. The limit of detection (LOD) was calculated according to IUPAC methodology which is the mean of the zero-ligand measurement added to three times its standard deviation₈₄.

### Detection of Endocrine Disrupting Chemicals (EDCs) in Human Samples Using Biosensor Protein.

Each CFPS reaction containing EDCs and human blood or urine was diluted 8-fold with PBS. This diluted CFPS reaction was further diluted with PBS and nitrocefin mixture (final 32- and 64-fold dilution for urine and blood, respectively). The final nitrocefin concentration was 200 mM. The activity of the reporter protein (β-lactamase) was measured as stated above. RNase inhibitor was added to each reaction at 32 U per 40 uL reaction.

### Results and Discussion

RAPID Biosensor Design for the hERβ.

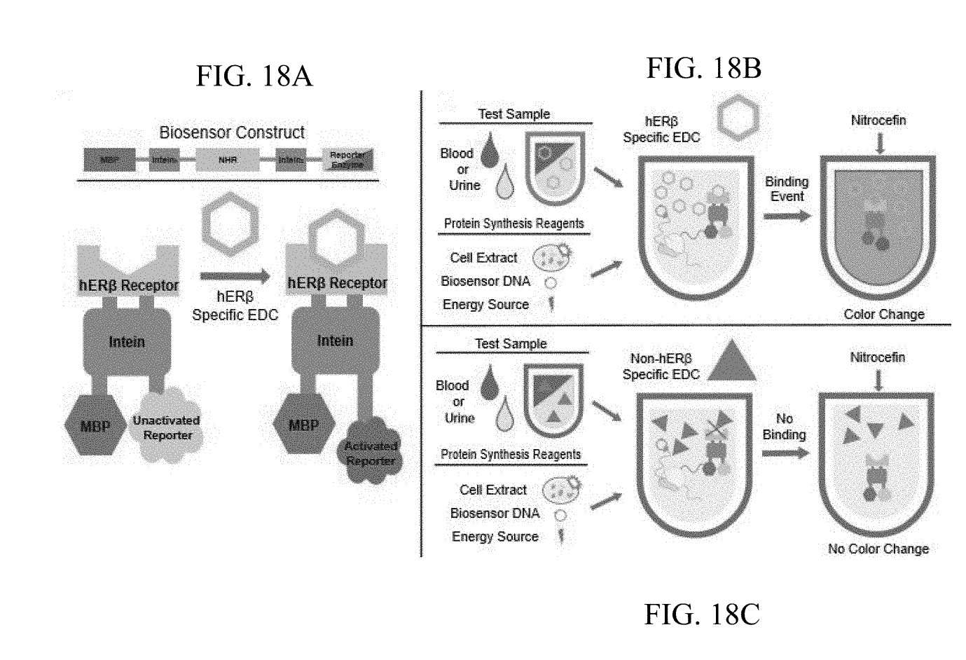

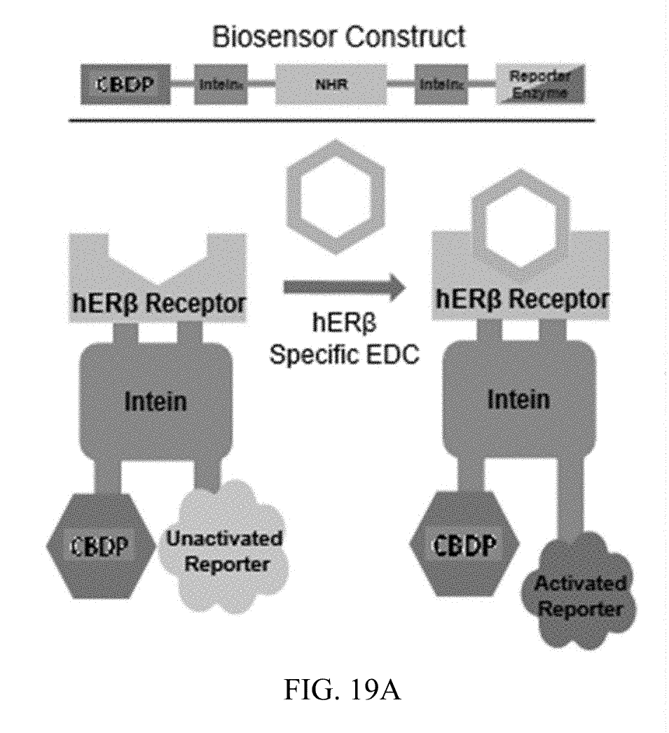

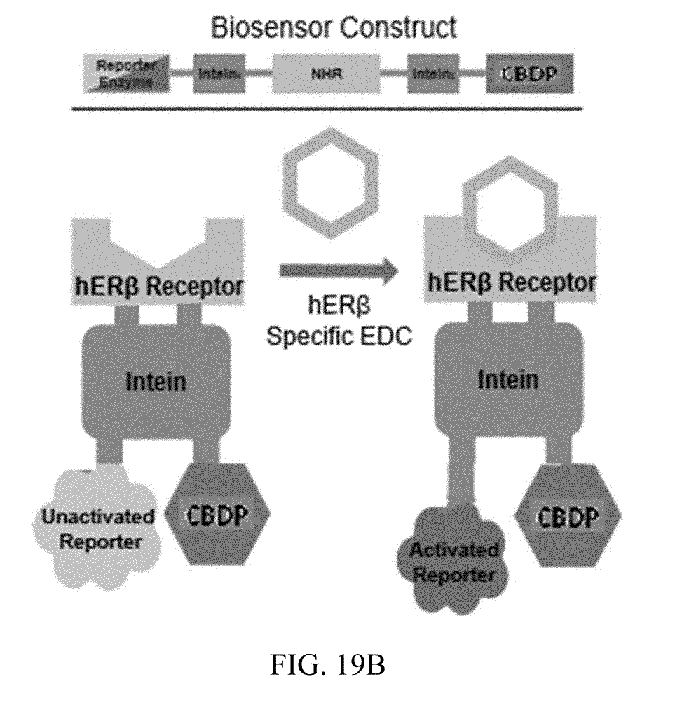

The modular, flexible nature of the RAPID biosensor is demonstrated for the first time by adapting it to detect hERβ-specific endocrine disruptors. The RAPID biosensor, as illustrated in FIG. 18A, was constructed by replacing the human thyroid receptor β ligand-binding domain in our previously reported sensor⁷⁹ with the hERβ ligand-binding domain. FIG. 18A part of a scheme RAPID biosensor assay and its mechanism of action, particularly the Estrogen receptor β RAPID biosensor construct with a MDP, where the human estrogen receptor β conformation changes upon interaction with hERβ-specific ligands. The conformation change transfers through the intein domain to the reporter enzyme (β-lactamase) which then becomes active. FIG. 19A shows alternative embodiments of the Estrogen receptor β RAPID biosensor construct and protein including a CBDP at its N-terminus, a mini-intein splicing domain with an inserted estrogen receptor β ligand binding domain, and a C-terminal reporter enzyme. FIG. 19B shows other alternative embodiments of the Estrogen receptor β RAPID biosensor construct and protein including a reporter enzyme at its N-terminus, a mini-intein splicing domain with an inserted estrogen receptor β ligand binding domain, and a C-terminal CBDP.

### Cell-Free Protein Synthesis of the Reporter Fusion Protein.

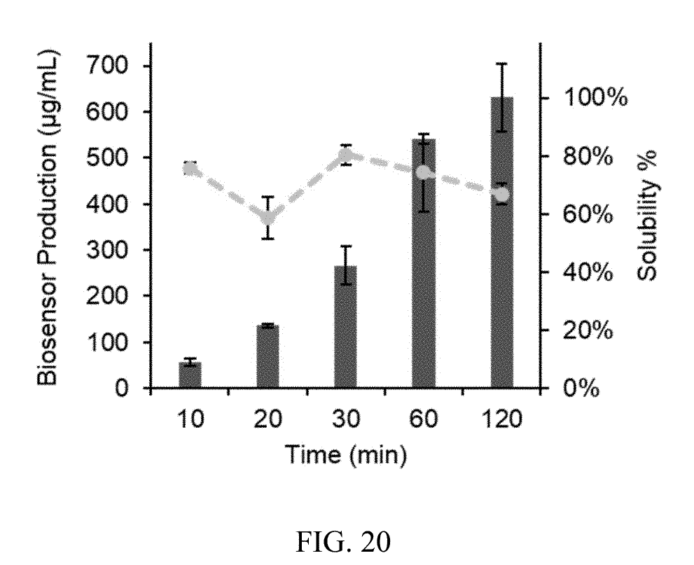

The RAPID biosensor protein (FIG. 18A) was expressed using an E. coli-based CFPS system. The total protein production and solubility percentage were measured by incorporating C-14 radiolabeled leucine as described in the method section. As illustrated in FIG. 20, the 120 kD RAPID biosensor protein was expressed up to 650 μg/mL in 3 hr with an average solubility of 75%. These results were similar to the hTRβ construct which achieved 700 μg/mL in 3 hrs of protein production⁷⁹. FIG. 20 shows the CFPS of the RAPID biosensor protein, particularly that the protein production yield increases with increasing reaction time. The solubility level remains relatively constant with increasing protein concentration. Bars represent total protein yield and dashed line represents solubility percentage. The error bars represent one standard deviation for n=3.

### RAPID Biosensor Assay.

The biosensor assay was performed as detailed in the methods section. Briefly, the two-step process includes: 1) expressing of the RAPID biosensor protein construct using CFPS in the sample to be tested and 2) performing nitrocefin colorimetric enzyme activity assay (FIGS. 18B and 18C). FIGS. 18B and 18C show part of a scheme RAPID biosensor assay and its mechanism of action, particularly the RAPID biosensor assay. The biosensor assay can be performed in the presence of human bodily samples (urine and blood). The presence of estrogen hERβ-specific ligands in the sample trigger a color change in the assay, which can be observed visually or more accurately measured using a spectrometer.

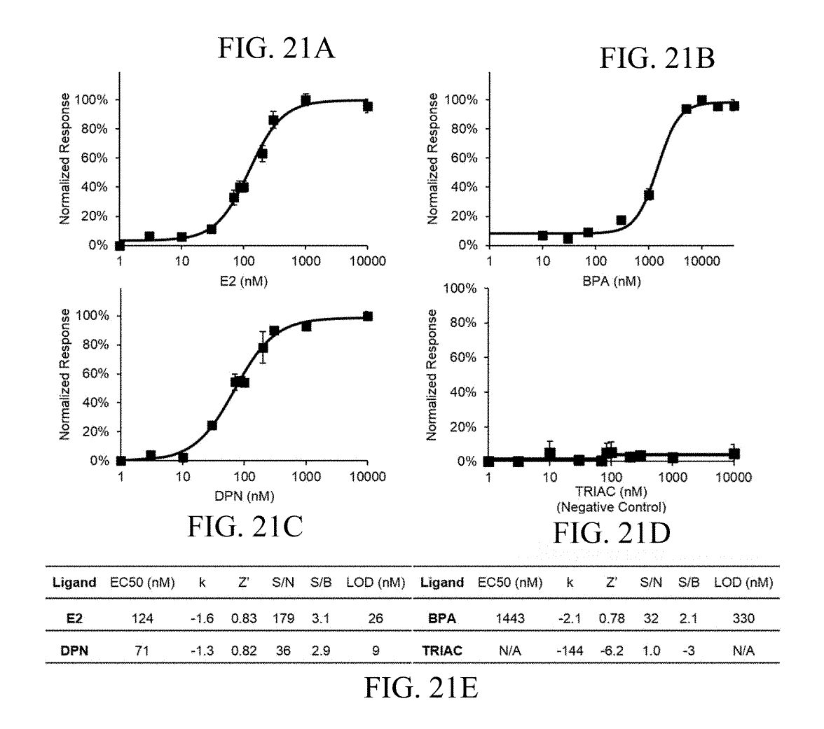

Biosensor performance was evaluated in the presence of four known endocrine disrupting compounds: E2, DPN, BPA, and TRIAC (FIG. 21A-21E). FIGS. 21A-21E show dose-response graphs and statistical analysis results for the Estrogen receptor β. RAPID biosensor in the presence of E2, DPN, BPA, and TRIAC (negative control, normalized based on the BPA maximum response). The EC₅₀ represents half-maximal effective concentration, k is the slope factor, the Z′ factor represents assay quality, S/N is the signal to noise ratio, S/B is the signal to background ratio, and LOD is the limit of detection. The solid line represents fitted nitrocefin conversion values, the square markers represent the measured values, and the error bars represent one standard deviation for n=2. E2, DPN, and BPA are known XEs (EDCs for the human estrogen nuclear hormone receptor) with different binding affinities, while TRIAC is a negative control (EDC for hTRβ). All assays with XEs produced dose-response graphs with excellent quality (Z′ factor was between 0.5 to 1.0). The EC₅₀ for E2, DPN, and BPA were 124, 71, and 1443 nM, respectively, which correspond well to the values obtained in our previous bacterial biosensor studies: 150, 190, and 1700 nM, respectively⁸². As expected the negative control, TRIAC, did not generate a statistically significant result, which is an indication of the estrogenic-chemical specificity of the assay. The total time to perform the biosensor assay was 2.5 hr (2 hr performing CFPS and 30 reading colorimetric assay), which is an order of magnitude less than the analogous cell based biosensors^{85,86,87}.

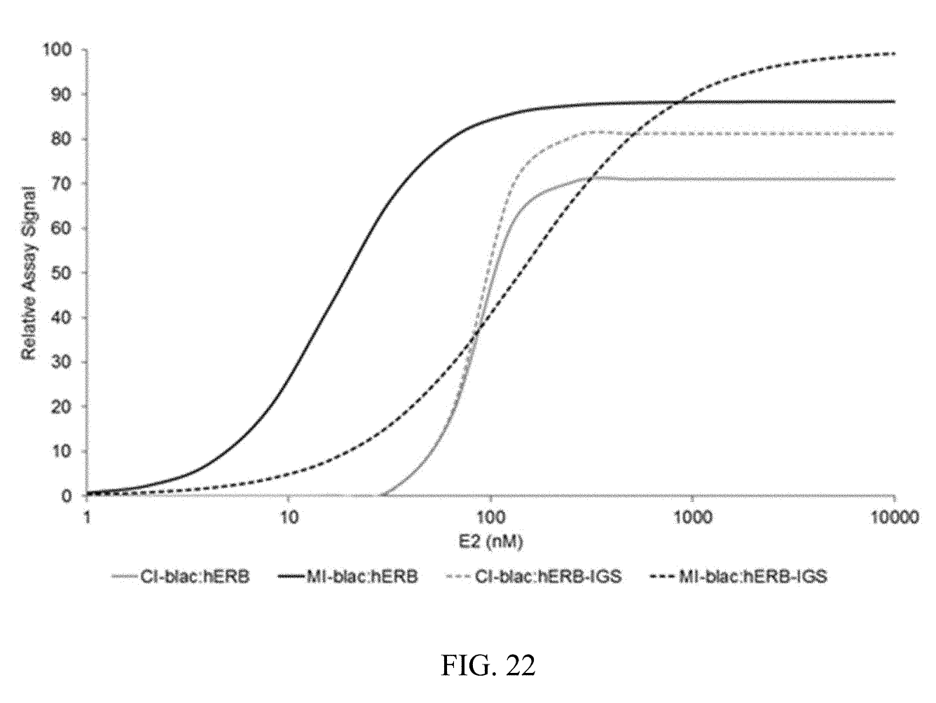

FIG. 22 also shows example dose responses of the estrogen receptor β biosensor proteins of various embodiments with MBP as shown in FIG. 18A (Black solid), with CBDP as shown in FIG. 19 (Gray solid), with MBP and a peptide linker (IGS) (Black dots), and with CBDP and a peptide linker (IGS) (Gray dots) in the presence of E2.

### Engineering CFPS for Human Bodily Samples.

To expand the applications of the biosensor, we previously showed that CFPS reactions maintain protein synthesis capability in the presence of various environmental samples including up to 45% by volume raw sewage. Although we illustrated that CFPS reactions can produce green florescent protein (GFP) in presence of human blood and urine, the protein yield was lower in these samples, especially for urine⁷⁹.

In this work, we engineered the CFPS reaction environment to improve protein yield in the presence of urine and blood. Although only small amounts of protein are necessary for the biosensor assay, increasing the protein yield reduces the quantity of reagents required which in turn reduces the cost of the biosensor.

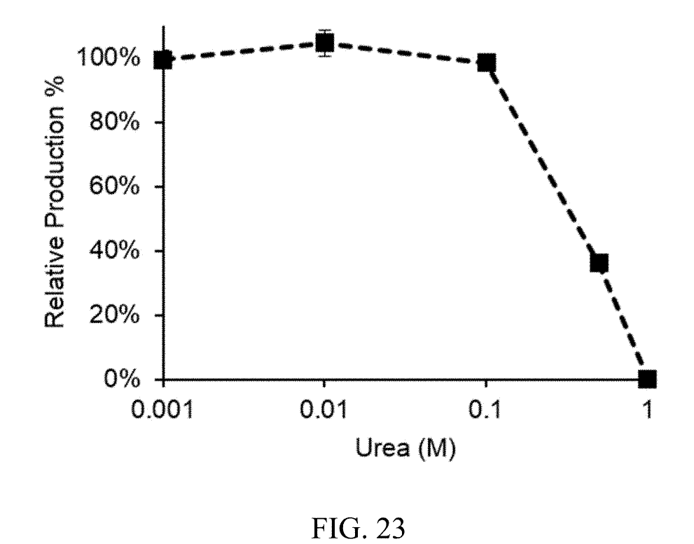

Initially, we hypothesized that urea is the main component to hinder protein synthesis in urine samples. The urea concentration in human samples is in a range of 0.1 to 0.8 M⁸⁸⁻⁸⁹ with the average being 0.22 M⁹⁰. To understand the effect of urea on CFPS, we synthesized the model protein GFP in the presence of urea at concentrations between 0.001 to 1 M (FIG. 23). FIG. 23 shows the effect of urea on CFPS of GFP. Error bars represent one standard deviation for n=3. The results revealed that 0.1 M urea doesn't have a significant effect on CFPS performance. Even reactions with 0.5 M urea retained 40% protein synthesis capability. We should point out that our previous work showed 10% urine, or about ˜0.02 M urea, effectively eliminated CFPS. These results suggested that urea is not the main component in urine to negatively impact the CFPS.

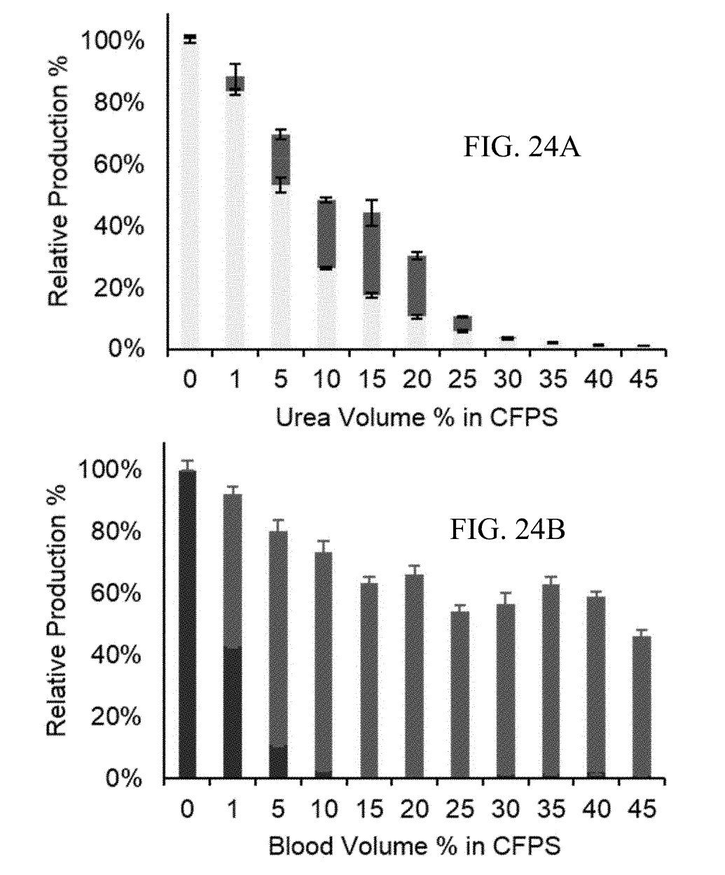

We next hypothesized that RNase activity hinders protein synthesis in both urine and blood samples^{91,92,93}. To test this hypothesis, we performed CFPS of GFP in the presence of urine and blood, and added murine RNase inhibitor as shown in FIGS. 24A and 24B. FIGS. 24A and 24B show the effect of RNase inhibitor on CFPS of GFP in the presence of urine (top) and blood (bottom). The lighter bars in both graphs represent the improvements in protein production in the presence of urine or blood with the addition of RNase inhibitor. The error bars represent one standard deviation for n=3. Results revealed significant improvement in protein synthesis. For instance, for 20% by volume urine sample, the GFP production yield increased from less than 10% to more than 30%. The improvement was more significant for blood samples, as 10% by volume blood eliminated protein synthesis, but even 45% by volume blood in CFPS containing RNase inhibitor retained 50% protein synthesis yield. The results suggest significant RNase activity in the blood samples and to a lesser extent in urine samples. Adding RNase inhibitors to CFPS reactions facilitates synthesis of sufficient biosensor protein to detect ligands in up to 25% and 45% by volume urine and blood, respectively. These higher volume percentages help to detect lower concentrations of ligands in the samples.

### RAPID Biosensor Performance in Human Urine and Blood.

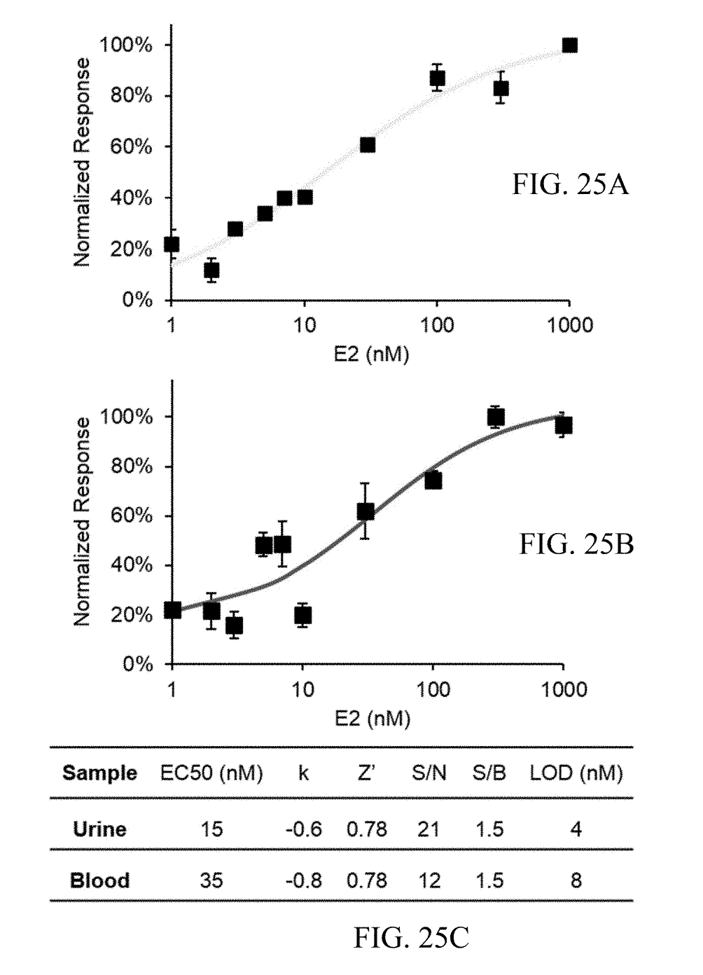

We chose 10% and 20% by volume of urine and blood, respectively, to investigate the robustness of the RNAse-inhibited biosensor assay in the presence of these human medical samples. As illustrated in FIG. 25A-25C, the biosensor assay generated a dose-response with EC₅₀ of 15 and 35 nM for E2 in presence of urine and blood, respectively. FIGS. 25A-25C show dose-response graph and statistical analysis results for the RAPID biosensor in the presence of E2 and 20% by volume blood and 10% by volume urine. RNAse inhibitors were added to both reactions. The solid line represents fitted nitrocefin conversion values, the square markers represent the measured values, and the error bars represent one standard deviation for n=2. The assay was deemed “excellent” for both samples with Z′=0.78 for both assays. The EC₅₀ is lower than the assay without urine and blood (FIGS. 21A-21E), which is like the result we observed for sensing TRIAC in raw sewage in our previous study. Additional engineering of the biosensor protein and CFPS reaction environment may enable increased sensitivity of the assay.

### Conclusion

Here we expanded the application of our RAPID biosensor to detect chemicals that interact with the human estrogen receptor β (XEs). We demonstrated the modular, flexible nature of this biosensor can be exploited to expand the RAPID biosensor to additional nuclear hormone receptors and the detection of other types of endocrine disrupting chemicals. The RAPID biosensor has the same level of sensitivity as the analogous E. coli cell-based assays and many other reported cell based methods, but achieves this result in a fraction of the time needed for these assays. In addition, we demonstrated for the first time that the RAPID biosensor can detect EDCs directly from human blood and urine, without sacrificing sensitivity, simply by adding RNAse inhibitors to the biosensor assay. Finally, we engineered the cell-free protein synthesis reaction environment to increase protein production yields, thereby ensuring increased assay sensitivity in the presence of blood and urine.

The following examples illustrate the various embodiments of the present invention. Those skilled in the art will recognize many variations that are within the spirit of the present invention and scope of the claims.

### Cell Extract Preparation

Yeast cell, mammalian cell, or bacterial cell extract is prepared in the following manner: 1. Cells are grown in culture to optimal concentration 2. Cells are harvested by centrifugation or other separation technique 3. Cells are lysed using a high-pressure homogenizer and cell extract is purified using centrifugation and/or size-exclusion chromatography 4. Cell extract is flash-frozen in liquid nitrogen and stored at −80° C.

### Cell-Free Protein Synthesis Reaction

Cell extract is combined with amino acids and cofactors, energetic molecules, and DNA coding for the nuclear hormone receptor and transcription cofactors. After an optimized reaction period during which soluble nuclear hormone receptors and cofactors are synthesized and properly folded, plasmid containing the appropriate hormone response element and a reporter protein such as beta-galactosidase is added to the reaction along with a reporter enzyme substrate, and a sample containing a nuclear hormone receptor modulator.

For portable/in-field biosensors, the nuclear hormone receptor is expressed in a cell-free protein synthesis reaction before genes coding for the reporter enzyme (under control of the hormone response element), an energy source, and a reporter enzyme substrate are added. The whole is then lyophilized to await reconstitution with an aqueous test sample of nuclear hormone receptor modulators.

### Biosensor Readout

As transcription of the reporter protein is activated, the reporter protein catalyzes a reaction with the enzymatic substrate. This reaction is quantified visually or with a spectrophotometer or other detector. For nuclear hormone receptor antagonizing modulators, the CFPS reaction is performed in the presence of a known agonist and the readout is obtained through a reduction in reporter protein activity.

### Molecular Mechanism of Action

Ligand-bound nuclear hormone receptors dimerize to form transcription activation complexes which bind to DNA sequences known as hormone response elements. This transcription holoenzyme recruits RNA polymerases which create mRNA. This mRNA is then translated into active protein by the protein synthesis machinery of the cell extract. The active protein then catalyzes a color-changing reaction which is interpreted as a nuclear hormone receptor-modulating signal.

The following references are incorporated herein by reference:
1. Nettles, K. W.; Greene, G. L. Annu. Rev. Physiol. 2005, 67, 309-333.
2. Bourguet, W.; Germain, P.; Gronemeyer, H. Trends Pharmacol. Sci. 2000, 21, 381-388.
3. Ohno, K.-i.; Fukushima, T.; Santa, T.; Waizumi, N.; Tokuyama, H.; Maeda, M.; Imai, K. Anal. Chem. 2002, 74, 4391-4396.
4. Hopkins, A. L.; Groom, C. R. Nature reviews Drug discovery 2002, 1, 727-730.
5. Fisher, J. S. Toxicology 2004, 205, 33-41.
6. Gore, A. C.; Chappell, V. A.; Fenton, S. E.; Flaws, J. A.; Nadal, A.; Prins, G. S.; Toppari, J.; Zoeller, R. T., Executive Summary to EDC-2: The Endocrine Society's Second Scientific Statement on Endocrine-Disrupting Chemicals. Endocr. Rev. 2015, 36 (6), 593-602.
7. Holmes, D., Endocrine disruptors: Air pollution linked to insulin resistance. Nat. Rev. Endocrinol. 2016, 12 (12), 688.
8. Aitkenhead, M. J.; Rhind, S. M.; Zhang, Z. L.; Kyle, C. E.; Coull, M. C., Neural network integration of field observations for soil endocrine disruptor characterisation. Sci. Total Environ. 2014, 468, 240-248.
9. Padhye, L. P.; Yao, H.; Kung′u, F. T.; Huang, C. H., Year-long evaluation on the occurrence and fate of pharmaceuticals, personal care products, and endocrine disrupting chemicals in an urban drinking water treatment plant. Water Res. 2014, 51, 266-76.
10. Minta, M.; Radko, L.; Stypula-Trebas, S.; Wozniak, B.; Zmudzki, J., Influence of dietary soy isoflavones on immature hamster uterotrophic and Hershberger assays. Bull. Vet. Inst. Pulawy 2013, 57 (4), 579-585.
11. De Coster, S.; van Larebeke, N., Endocrine-disrupting chemicals: associated disorders and mechanisms of action. J. Environ. Public Health 2012, 2012, 713696.
12. Scognamiglio, V.; Antonacci, A.; Patrolecco, L.; Lambreva, M. D.; Litescu, S. C.; Ghuge, S. A.; Rea, G., Analytical tools monitoring endocrine disrupting chemicals. Trac-Trends Anal. Chem. 2016, 80, 555-567.
13. Soto, A. M.; Sonnenschein, C., Environmental causes of cancer: endocrine disruptors as carcinogens. Nat. Rev. Endocrinol. 2010, 6 (7), 363-70.
14. Attina, T. M.; Hauser, R.; Sathyanarayana, S.; Hunt, P. A.; Bourguignon, J. P.; Myers, J. P.; DiGangi, J.; Zoeller, R. T.; Trasande, L., Exposure to endocrine-disrupting chemicals in the USA: a population-based disease burden and cost analysis. Lancet Diabetes Endocrinol. 2016, 4 (12), 996-1003.
15. Nalbone, G.; Cicolella, A.; Laot-Cabon, S., Endocrine disruptors and metabolic diseases: a major public health challenge. Sante Publique 2013, 25 (1), 45-49.
16. Philips, E. M.; Jaddoe, V. W. V.; Trasande, L., Effects of early exposure to phthalates and bisphenols on cardiometabolic outcomes in pregnancy and childhood. Reprod. Toxicol. 2017, 68, 105-118.
17. Teixeira, D.; Pestana, D.; Santos, C.; Correia-Sá, L.; Marques, C.; Norberto, S.; Meireles, M.; Faria, A.; Silva, R.; Faria, G., Inflammatory and cardiometabolic risk on obesity: role of environmental xenoestrogens. The J. Clin. Endocrinol. Metab. 2015, 100 (5), 1792-1801.
18. Titus-Ernstoff, L.; Troisi, R.; Hatch, E. E.; Palmer, J. R.; Hyer, M.; Kaufman, R.; Adam, E.; Noller, K.; Hoover, R. N., Birth defects in the sons and daughters of women who were exposed in utero to diethylstilbestrol (DES). Int. J. Androl. 2010, 33 (2), 377-383.
19. Vilahur, N.; Bustamante, M.; Byun, H.-M.; Fernandez, M. F.; Santa Marina, L.; Basterrechea, M.; Ballester, F.; Murcia, M.; Tardón, A.; Fernández-Somoano, A., Prenatal exposure to mixtures of xenoestrogens and repetitive element DNA methylation changes in human placenta. Environ. Int. 2014, 71, 81-87.
20. Morgan, M.; Deoraj, A.; Felty, Q.; Roy, D., Environmental estrogen-like endocrine disrupting chemicals and breast cancer. Mol. Cell. Endocrinol. 2016.
21. N'Tumba-Byn, T.; Moison, D.; Lacroix, M.; Lecureuil, C.; Lesage, L.; Prud'homme, S. M.; Pozzi-Gaudin, S.; Frydman, R.; Benachi, A.; Livera, G.; Rouiller-Fabre, V.; Habert, R., Differential Effects of Bisphenol A and Diethylstilbestrol on Human, Rat and Mouse Fetal Leydig Cell Function. PLoS One 2012, 7 (12), 10.
22. Zwart, W.; Terra, H.; Linn, S. C.; Schagen, S. B., Cognitive effects of endocrine therapy for breast cancer: keep calm and carry on? Nat. Rev. Clin. Oncol. 2015, 12 (10), 597-606.
23. Elsworth, J. D.; Jentsch, J. D.; Groman, S. M.; Roth, R. H.; Redmond, D. E., Jr.; Leranth, C., Low Circulating Levels of Bisphenol-A Induce Cognitive Deficits and Loss of Asymmetric Spine Synapses in Dorsolateral Prefrontal Cortex and Hippocampus of Adult Male Monkeys. J. Comp. Neurol. 2015, 523 (8), 1248-1257.
24. Vilahur, N.; Fernández, M. F.; Bustamante, M.; Ramos, R.; Forns, J.; Ballester, F.; Murcia, M.; Riaño, I.; Ibarluzea, J.; Olea, N., In utero exposure to mixtures of xenoestrogens and child neuropsychological development. Environ. Res. 2014, 134, 98-104.
25. Moens, S. J. B.; Schnitzler, G. R.; Nickerson, M.; Guo, H.; Ueda, K.; Lu, Q.; Aronovitz, M. J.; Nickerson, H.; Baur, W. E.; Hansen, U., Rapid estrogen receptor signaling is essential for the protective effects of estrogen against vascular injury. Circulation 2012, 126 (16), 1993-2004.
26. Johansen, L. M.; Brannan, J. M.; Delos, S. E.; Shoemaker, C. J.; Stossel, A.; Lear, C.; Hoffstrom, B. G.; DeWald, L. E.; Schomberg, K. L.; Scully, C., FDA-approved selective estrogen receptor modulators inhibit Ebola virus infection. Sci. Transl. Med. 2013, 5 (190).
27. Hormann, A. M.; vom Saal, F. S.; Nagel, S. C.; Stahlhut, R. W.; Moyer, C. L.; Ellersieck, M. R.; Welshons, W. V.; Toutain, P. L.; Taylor, J. A., Holding Thermal Receipt Paper and Eating Food after Using Hand Sanitizer Results in High Serum Bioactive and Urine Total Levels of Bisphenol A (BPA). PLoS One 2014, 9 (10), 12.
28. Venners, S. A.; Liu, X.; Perry, M. J.; Korrick, S. A.; Li, Z. P.; Yang, F.; Yang, J. H.; Lasley, B. L.; Xu, X. P.; Wang, X. B., Urinary estrogen and progesterone metabolite concentrations in menstrual cycles of fertile women with non-conception, early pregnancy loss or clinical pregnancy. Hum. Reprod. 2006, 21 (9), 2272-2280.
29. Leusch, F. D. L.; De Jager, C.; Levi, Y.; Lim, R.; Puijker, L.; Sacher, F.; Tremblay, L. A.; Wilson, V. S.; Chapman, H. F., Comparison of Five in Vitro Bioassays to Measure Estrogenic Activity in Environmental Waters. Environ. Sci. Technol. 2010, 44 (10), 3853-3860.
30. Routledge, E. J.; Sumpter, J. P., Estrogenic activity of surfactants and some of their degradation products assessed using a recombinant yeast screen. Environ. Toxicol. Chem. 1996, 15 (3), 241-248.
31. Soto, A. M.; Sonnenschein, C.; Chung, K. L.; Fernandez, M. F.; Olea, N.; Serrano, F. O., The E-SCREEN assay as a tool to identify estrogens: an update on estrogenic environmental pollutants. Environ. Health Perspect. 1995, 103 Suppl 7, 113-22.
32. Legler, J.; van den Brink, C. E.; Brouwer, A.; Murk, A. J.; van der Saag, P. T.; Vethaak, A. D.; van der Burg, P., Development of a stably transfected estrogen receptor-mediated luciferase reporter gene assay in the human T47D breast cancer cell line. Toxicol. Sci. 1999, 48 (1), 55-66.
33. Balaguer, P.; Francois, F.; Comunale, F.; Fenet, H.; Boussioux, A. M.; Pons, M.; Nicolas, J. C.; Casellas, C., Reporter cell lines to study the estrogenic effects of xenoestrogens. Sci. Total Environ. 1999, 233 (1-3), 47-56.
34. Sonneveld, E.; Jansen, H. J.; Riteco, J. A.; Brouwer, A.; van der Burg, B., Development of androgen- and estrogen-responsive bioassays, members of a panel of human cell line-based highly selective steroid-responsive bioassays. Toxicol. Sci. 2005, 83 (1), 136-48.
35. Brennan, J. C.; Bassal, A.; He, G.; Denison, M. S., Development of a recombinant human ovarian (BG1) cell line containing estrogen receptor alpha and beta for improved detection of estrogenic/antiestrogenic chemicals (vol 35, pg 91, 2016). Environ. Toxicol. Chem. 2017, 36 (5), 1405-1405.
36. Alvarez, D. A.; Shappell, N. W.; Billey, L. O.; Bermudez, D. S.; Wilson, V. S.; Kolpin, D. W.; Perkins, S. D.; Evans, N.; Foreman, W. T.; Gray, J. L.; Shipitalo, M. J.; Meyer, M. T., Bioassay of estrogenicity and chemical analyses of estrogens in streams across the United States associated with livestock operations. Water Res. 2013, 47 (10), 3347-3363.
37. Conley, J. M.; Evans, N.; Mash, H.; Rosenblum, L.; Schenck, K.; Glassmeyer, S.; Furlong, E. T.; Kolpin, D. W.; Wilson, V. S., Comparison of in vitro estrogenic activity and estrogen concentrations in source and treated waters from 25 U.S. drinking water treatment plants. Sci. Total Environ. 2017, 579, 1610-1617.
38. Ye, X.; Kuklenyik, Z.; Needham, L. L.; Calafat, A. M., Automated on-line column-switching HPLC-MS/MS method with peak focusing for the determination of nine environmental phenols in urine. Anal. Chem. 2005, 77 (16), 5407-5413.
39. Covaci, A.; Den Hond, E.; Geens, T.; Govarts, E.; Koppen, G.; Frederiksen, H.; Knudsen, L. E.; Mørck, T. A.; Gutleb, A. C.; Guignard, C., Urinary BPA measurements in children and mothers from six European member states: overall results and determinants of exposure. Environ. Res. 2015, 141, 77-85.
40. Tomaszewski, M.; White, C.; Patel, P.; Masca, N.; Damani, R.; Hepworth, J.; Samani, N. J.; Gupta, P.; Madira, W.; Stanley, A., High rates of non-adherence to antihypertensive treatment revealed by high-performance liquid chromatography-tandem mass spectrometry (HP LC-MS/MS) urine analysis. Heart 2014, 305063.
41. McLachlan, M. J.; Katzenellenbogen, J. A.; Zhao, H., A new fluorescence complementation biosensor for detection of estrogenic compounds. Biotechnol. Bioeng. 2011, 108 (12), 2794-803.
42. De, S.; Macara, I. G.; Lannigan, D. A., Novel biosensors for the detection of estrogen receptor ligands. J. Steroid Biochem. Mol. Biol. 2005, 96 (3-4), 235-244.
43. Skretas, G.; Wood, D. W. J. Mol. Biol. 2005, 349, 464-474.
44. Skretas, G.; Meligova, A. K.; Villalonga-Barber, C.; Mitsiou, D. J.; Alexis, M. N.; Micha-Screttas, M.; Steele, B. R.; Screttas, C. G.; Wood, D. W. J. Am. Chem. Soc. 2007, 129, 8443-8457.
45. Gierach, I.; Li, J.; Wu, W.-Y.; Grover, G. J.; Wood, D. W. FEBS open bio 2012, 2, 247-253.
46. Gierach, I.; Shapero, K.; Eyster, T. W.; Wood, D. W. Environ. Toxicol. 2013, 28, 179-189.
47. Shi, C.; Meng, Q.; Wood, D. W. J. Mol. Endocrinol. 2014, 52, 55-66.
48. Chen, T.; Xie, W.; Agler, M.; Banks, M. Assay Drug Dev. Technol. 2003, 1, 835-842.
49. Vamvakaki, V.; Tsagaraki, K.; Chaniotakis, N. Anal. Chem. 2006, 78, 5538-5542.
50. Bahadir, E. B.; Sezgintürk, M. K. Anal. Biochem. 2015, 478, 107-120.
51. Ziegler, C. Fresenius J. Anal. Chem. 2000, 366, 552-559.
52. Liu, Q.; Wu, C.; Cal, H.; Hu, N.; Zhou, J.; Wang, P. Chem. Rev. 2014, 114, 6423-6461.
53. Salehi, A. S.; Earl, C. C.; Muhlestein, C.; Bundy, B. C. Int. J. Dev. Biol. 2016.
54. Pardee, K.; Green, Alexander A.; Ferrante, T.; Cameron, D. E.; DaleyKeyser, A.; Yin, P.; Collins, James J. Cell 2014, 159, 940-954.
55. Schinn, S.-M.; Broadbent, A.; Bradley, W. T.; Bundy, B. C. New Biotechnology 2016, 33, 480-487.
56. Gierach, I.; Li, J.; Wu, W.-Y.; Grover, G. J.; Wood, D. W. FEBS open bio 2012, 2, 247-253.
57. Banki, M. R.; Feng, L.; Wood, D. W. Nat Methods 2005, 2, 659-661.
58. Shrestha, P.; Holland, T. M.; Bundy, B. C. Biotechniques 2012, 53, 12.
59. Smith, M. T.; Berkheimer, S. D.; Werner, C. J.; Bundy, B. C. Biotechniques 2014, 56, 186-193.
60. Smith, M. T.; Bennett, A. M.; Hunt, J. M.; Bundy, B. C. Biotechnol. Prog. 2015, 31, 1716-1719.
61. Jewett, M. C.; Swartz, J. R. Biotechnol. Bioeng. 2004, 86, 19-26.
62. Wuu, J. J.; Swartz, J. R. Biochim. Biophys. Acta 2008, 1778, 1237-1250.
63. Bourgeois, S.; Gernet, M.; Pradeau, D.; Andremont, A.; Fattal, E. Int. J. Pharm. 2006, 324, 2-9.
64. O'Connell, M. A.; Belanger, B. A.; Haaland, P. D. Chemometrics Intellig. Lab. Syst. 1993, 20, 97-114.
65. Gierach, I.; Shapero, K.; Eyster, T. W.; Wood, D. W. Environ. Toxicol. 2013, 28, 179-189.
66. Zhang, J.-H.; Chung, T. D.; Oldenburg, K. R. J. Biomol. Screen. 1999, 4, 67-73.
67. Vareiro, M. M. L. M.; Liu, J.; Knoll, W.; Zak, K.; Williams, D.; Jenkins, A. T. A. Anal. Chem. 2005, 77, 2426-2431.
68. Smith, M. T.; Wilding, K. M.; Hunt, J. M.; Bennett, A. M.; Bundy, B. C. FEBS Lett. 2014, 588, 2755-2761.
69. Skretas, G.; Wood, D. W. Protein Sci. 2005, 14, 523-532.
70. Swartz, J. R. AIChE J. 2012, 58, 5-13.
71. Martínez, L.; Nascimento, A. S.; Nunes, F. M.; Phillips, K.; Aparicio, R.; Dias, S. M. G.; Figueira, A. C. M.; Lin, J. H.; Nguyen, P.; Apriletti, J. W. Proceedings of the National Academy of Sciences 2009, 106, 20717-20722.
72. Takeda, T.; Suzuki, S.; Liu, R.; DeGroot, L. J. The Journal of Clinical Endocrinology & Metabolism 1995, 80, 2033-2040.
73. Marchesini, G. R.; Meulenberg, E.; Haasnoot, W.; Mizuguchi, M.; Irth, H. Anal. Chem. 2006, 78, 1107-1114.
74. Borgert, C. J.; Mihaich, E. M.; Quill, T. F.; Marty, M. S.; Levine, S. L.; Becker, R. A. Regul. Toxicol. Pharmacol. 2011, 59, 397-411.
75. Zacharewski, T. Environ. Sci. Technol. 1997, 31, 613-623.
76. Gierach, I.; Wood, D. W. In Biosensors for Health, Environment and Biosecurity, Serra, P. A., Ed.; InTech, 2011.
77. Salehi, A. S. M.; Smith, M. T.; Bennett, A. M.; Williams, J. B.; Pitt, W. G.; Bundy, B. C. Biotechnology Journal 2015, n/a-n/a.
78. Ipe, D. S.; Horton, E.; Ulett, G. C. Frontiers in cellular and infection microbiology 2016, 6.
79. Salehi, A. S. M.; Tang, M. J. S.; Smith, M. T.; Hunt, J. M.; Law, R. A.; Wood, D. W.; Bundy, B. C., Cell-Free Protein Synthesis Approach to Biosensing hTRβ-Specific Endocrine Disruptors. Anal. Chem. 2017, 89 (6), 3395-3401.
80. Smith, M. T.; Hawes, A. K.; Shrestha, P.; Rainsdon, J. M.; Wu, J. C.; Bundy, B. C., Alternative fermentation conditions for improved Escherichia coli-based cell-free protein synthesis for proteins requiring supplemental components for proper synthesis. Process Biochem. 2014, 49 (2), 217-222.
81. Salehi, A. S. M.; Smith, M. T.; Bennett, A. M.; Williams, J. B.; Pitt, W. G.; Bundy, B. C., Cell-free protein synthesis of a cytotoxic cancer therapeutic: Onconase production and a just-add-water cell-free system. Biotechnol. J. 2016, 11 (2), 274-281.
82. Gierach, I.; Shapero, K.; Eyster, T. W.; Wood, D. W., Bacterial biosensors for evaluating potential impacts of estrogenic endocrine disrupting compounds in multiple species. Environ. Toxicol. 2013, 28 (4), 179-89.
83. Zhang, J. H.; Chung, T. D. Y.; Oldenburg, K. R., A simple statistical parameter for use in evaluation and validation of high throughput screening assays. J. Biomol. Screen. 1999, 4 (2), 67-73.
84. Vareiro, M. L. M.; Liu, J.; Knoll, W.; Zak, K.; Williams, D.; Jenkins, A. T. A., Surface plasmon fluorescence measurements of human chorionic gonadotrophin: Role of antibody orientation in obtaining enhanced sensitivity and limit of detection. Anal. Chem. 2005, 77 (8), 2426-2431.
85. Skretas, G.; Wood, D. W., A bacterial biosensor of endocrine modulators. J. Mol. Biol. 2005, 349 (3), 464-474.
86. Skretas, G.; Meligova, A. K.; Villalonga-Barber, C.; Mitsiou, D. J.; Alexis, M. N.; Micha-Screttas, M.; Steele, B. R.; Screttas, C. G.; Wood, D. W., Engineered chimeric enzymes as tools for drug discovery: generating reliable bacterial screens for the detection, discovery, and assessment of estrogen receptor modulators. J. Am. Chem. Soc. 2007, 129 (27), 8443-57.
87. Gierach, I.; Li, J.; Wu, W.-Y.; Grover, G. J.; Wood, D. W., Bacterial biosensors for screening isoform-selective ligands for human thyroid receptors alpha-1 and beta-1. FEBS Open Bio 2012, 2, 247-253.
88. Shaykhutdinov, R., Maclnnis, G., Dowlatabadi, R., Weljie, A., Vogel, H., Quantitative analysis of metabolite concentrations in human urine samples using 13C{1H} NMR spectroscopy. Metabolomics 2009, 5 (3), 307-317.
89. Taylor, A. J.; Vadgama, P., Analytical reviews in clinical biochemistry: the estimation of urea. Ann. Clin. Biochem. 1992, 29 (Pt 3), 245-64.
90. Putnam, D. F. Composition and concentrative properties of human urine; CF-1802; NASA: Washington, D C, 1971.
91. Blank, A.; Dekker, C. A., Ribonucleases of human serum, urine, cerebrospinal fluid, and leukocytes. Activity staining following electrophoresis in sodium dodecyl sulfate-polyacrylamide gels. Biochemistry 1981, 20 (8), 2261-7.
92. Iwama, M.; Kunihiro, M.; Ohgi, K.; Irie, M., Purification and properties of human urine ribonucleases. J. Biochem. 1981, 89 (4), 1005-16.
93. Landre, J. B.; Hewett, P. W.; Olivot, J. M.; Friedl, P.; Ko, Y.; Sachinidis, A.; Moenner, M., Human endothelial cells selectively express large amounts of pancreatic-type ribonuclease (RNase 1). J. Cell. Biochem. 2002, 86 (3), 540-52.

While exemplary embodiments are described above, it is not intended that these embodiments describe all possible forms of the invention. Rather, the words used in the specification are words of description rather than limitation, and it is understood that various changes may be made without departing from the spirit and scope of the invention. Additionally, the features of various implementing embodiments may be combined to form further embodiments of the invention.

What is claimed is:

1. A cell-free method of detecting compounds in a sample comprising the step of expressing biosensor proteins with a cell extract and in the presence of a sample, wherein the biosensor proteins during expressing bind compounds in the sample and are capable of generating detectable signals only when the biosensor proteins bind ligand during expressing.

2. The method of claim 1, wherein the expressing includes folding the biosensor proteins.

3. The method of claim 1, wherein the cell extract is without viable cells.

4. The method of claim 1, wherein the cell extract is a prokaryotic cell extract or eukaryotic extract.

5. The method of claim 1, wherein the biosensor proteins are expressed with a lyophilized cell extract.

6. The method of claim 1, wherein the sample is a biological sample.

7. The method of claim 6, wherein the biological sample is a sample from an individual including at least one of whole blood, serum, plasma, urine, feces, sputum, saliva, or sweat.

8. The method of claim 1, wherein the sample is an environmental sample.

9. The method of claim 8, wherein the environmental sample is a water sample, a tap water sample, a rain/storm water sample, a snow sample, a waste water sample, or water sample from a pond, lake, or river, a soil sample, a sewage sample, a raw sewage sample, a post clarifier sample, a post biological sample, a post filter sample, or an effluent sample.

10. A cell-free method of screening compounds for pharmaceutical applications comprising the step of expressing biosensor proteins with a cell extract and in the presence of a test compound, wherein the biosensor proteins are capable of generating detectable signals only when the biosensor proteins bind the test compound during expressing and the test compound is identified for a pharmaceutical application if it binds to the biosensor proteins.

11. The method of claim 10, wherein the test compound is identified as an antagonist to ligand-binding to or an agonist of a receptor if the test compound binds to the biosensor proteins.

12. The method of claim 10, wherein the for pharmaceutical applications are as nuclear hormone modulators and the test compound is identified as a nuclear hormone modulator if it binds to the biosensor proteins.

13. The method of claim 10, wherein the expressing includes folding the biosensor proteins.

14. The method of claim 10, wherein the biosensor proteins are expressed with a lyophilized cell extract.

15. The method of claim 10, wherein the cell extract is without viable cells.

16. The method of claim 10, wherein the cell extract is a prokaryotic cell extract or eukaryotic extract.

17. A kit comprising:
an expression cassette including a polynucleotide encoding for a biosensor protein; and
a cell extract that is capable of being combined with at least the expression cassette and a sample or test compound in a cell-free reaction medium for expressing biosensor proteins.

18. The kit of claim 17, wherein the cell extract is a lyophilized cell extract.

19. The kit of claim 17, wherein the cell extract is a prokaryotic cell extract or eukaryotic extract.

20. The kit of claim 17, wherein the cell extract is without viable cells.


SEQUENCE LISTING (8 sequences)

SEQ ID NO: 1 - DNA, 3267 bp, Artificial
  Polynucleotide Sequence Encoding Thyroid Receptor Beta Biosensor Protein Including Maltose Binding Protein
atgaaaatcgaagaaggtaaactggtaatctggattaacggcgataaaggctataacggtctcgctgaagtcggtaagaaattcgagaaagataccggaattaaagtcaccgttgagcatccggataaactggaagagaaattcccacaggttgcggcaactggcgatggccctgacattatcttctgggcacacgaccgctttggtggctacgctcaatctggcctgttggctgaaatcaccccggacaaagcgttccaggacaagctgtatccgtttacctgggatgccgtacgttacaacggcaagctgattgcttacccgatcgctgttgaagcgttatcgctgatttataacaaagatctgctgccgaacccgccaaaaacctgggaagagatcccggcgctggataaagaactgaaagcgaaaggtaagagcgcgctgatgttcaacctgcaagaaccgtacttcacctggccgctgattgctgctgacgggggttatgcgttcaagtatgaaaacggcaagtacgacattaaagacgtgggcgtggataacgctggcgcgaaagcgggtctgaccttcctggttgacctgattaaaaacaaacacatgaatgcagacaccgattactccatcgcagaagctgcctttaataaaggcgaaacagcgatgaccatcaacggcccgtgggcatggtccaacatcgacaccagcaaagtgaattatggtgtaacggtactgccgaccttcaagggtcaaccatccaaaccgttcgttggcgtgctgagcgcaggtattaacgccgccagtccgaacaaagagctggcaaaagagttcctcgaaaactatctgctgactgatgaaggtctggaagcggttaataaagacaaaccgctgggtgccgtagcgctgaagtcttacgaggaagagttggcgaaagatccacgtattgccgccaccatggaaaacgcccagaaaggtgaaatcatgccgaacatcccgcagatgtccgctttctggtatgccgtgcgtactgcggtgatcaacgccgccagcggtcgtcagactgtcgatgaagccctgaaagacgcgcagactaattcgagctcgaacaacaacaacaataacaataacaacaacctcgggatcgagggaaggatttcagaattcgccctcgcagagggcactcggatcttcgatccggtcaccggtacaacgcatcgcatcgaggatgttgtcgatgggcgcaagcctattcatgtcgtggctgctgccaaggacggaacgctgcatgcgcggcccgtggtgtcctggttcgaccagggaacgcgggatgtgatcgggttgcggatcgccggtggcgccatcgtgtgggcgacacccgatcacaaggtgctgacagagtacggctggcgtgccgcgggggaactccgcaagggagacagggtggcgcaaccgcgacgcttcgatggattcggtgacagtgcgccgattccggcgcgcctgcagaagtccatcgggcacaagccagagcccacagacgaggaatgggagctcatcaaaactgtcaccgaagcccatgtggcgaccaacgcccaaggcagccactggaagcaaaaacggaaattcctgccagaagacattggacaagcaccaatagtcaatgccccagaaggtggaaaggttgacttggaagccttcagccattttacaaaaatcatcacaccagcaattaccagagtggtggattttgccaaaaagttgcctatgttttgtgagctgccatgtgaagaccagatcatcctcctcaaaggctgctgcatggagatcatgtcccttcgcgctgctgtgcgctatgacccagaaagtgagactttaaccttgaatggggaaatggcagtgacacggggccagctgaaaaatgggggtcttggggtggtgtcagacgccatctttgacctgggcatgtctctgtcttctttcaacctggatgacactgaagtagccctccttcaggccgtcctgctgatgtcttcagatcgcccggggcttgcctgtgttgagagaatagaaaagtaccaagatagtttcctgctggcctttgaacactatatcaattaccgaaaacaccacgtgacacacttttggccaaaactcctgatgaaggtgacagatctgcggatgataggagcctgccatgccagccgcttcctgcacatgaaggtggaatgccccacagaactcttcccccctttgttcttggaagtgttcgaggatgcggatgccctggatgacaaattcctgcacgacatgctggcggaagaactccgctattccgtgatccgagaagtgctgccaacgcggcgggcacgaacgttcgacctcgaggtcgaggaactgcacaccctcgtcgccgaaggggttgttgtacacaacatgcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattgggcggccgcactcgagcaccaccaccaccaccactga

SEQ ID NO: 2 - protein, 1077 aa, Artificial
  Amino Acid Sequence Of Thyroid Receptor Beta Biosensor Protein Including Maltose Binding Protein
MKIEEGKLVIWINGDKGYNGLAEVGKKFEKDTGIKVTVEHPDKLEEKFPQVAATGDGPDIIFWAHDRFGGYAQSGLLAEITPDKAFQDKLYPFTWDAVRYNGKLIAYPIAVEALSLIYNKDLLPNPPKTWEEIPALDKELKAKGKSALMFNLQEPYFTWPLIAADGGYAFKYENGKYDIKDVGVDNAGAKAGLTFLVDLIKNKHMNADTDYSIAEAAFNKGETAMTINGPWAWSNIDTSKVNYGVTVLPTFKGQPSKPFVGVLSAGINAASPNKELAKEFLENYLLTDEGLEAVNKDKPLGAVALKSYEEELAKDPRIAATMENAQKGEIMPNIPQMSAFWYAVRTAVINAASGRQTVDEALKDAQTNSSSNNNNNNNNNNLGIEGRISEFALAEGTRIFDPVTGTTHRIEDVVDGRKPIHVVAAAKDGTLHARPVVSWFDQGTRDVIGLRIAGGAIVWATPDHKVLTEYGWRAAGELRKGDRVAQPRRFDGFGDSAPIPARLQKSIGHKPEPTDEEWELIKTVTEAHVATNAQGSHWKQKRKFLPEDIGQAPIVNAPEGGKVDLEAFSHFTKIITPAITRVVDFAKKLPMFCELPCEDQIILLKGCCMEIMSLRAAVRYDPESETLTLNGEMAVTRGQLKNGGLGVVSDAIFDLGMSLSSFNLDDTEVALLQAVLLMSSDRPGLACVERIEKYQDSFLLAFEHYINYRKHHVTHFWPKLLMKVTDLRMIGACHASRFLHMKVECPTELFPPLFLEVFEDADALDDKFLHDMLAEELRYSVIREVLPTRRARTFDLEVEELHTLVAEGVVVHNMHPETLVKVKDAEDQLGARVGYIELDLNSGKILESFRPEERFPMMSTFKVLLCGAVLSRIDAGQEQLGRRIHYSQNDLVEYSPVTEKHLTDGMTVRELCSAAITMSDNTAANLLLTTIGGPKELTAFLHNMGDHVTRLDRWEPELNEAIPNDERDTTMPVAMATTLRKLLTGELLTLASRQQLIDWMEADKVAGPLLRSALPAGWFIADKSGAGERGSRGIIAALGPDGKPSRIVVIYTTGSQATMDERNRQIAEIGASLIKHW

SEQ ID NO: 3 - DNA, 3261 bp, Artificial
  Polynucleotide Sequence Encoding Estrogen Receptor Beta Biosensor Protein Including Maltose Binding Protein
atgaaaatcgaagaaggtaaactggtaatctggattaacggcgataaaggctataacggtctcgctgaagtcggtaagaaattcgagaaagataccggaattaaagtcaccgttgagcatccggataaactggaagagaaattcccacaggttgcggcaactggcgatggccctgacattatcttctgggcacacgaccgctttggtggctacgctcaatctggcctgttggctgaaatcaccccggacaaagcgttccaggacaagctgtatccgtttacctgggatgccgtacgttacaacggcaagctgattgcttacccgatcgctgttgaagcgttatcgctgatttataacaaagatctgctgccgaacccgccaaaaacctgggaagagatcccggcgctggataaagaactgaaagcgaaaggtaagagcgcgctgatgttcaacctgcaagaaccgtacttcacctggccgctgattgctgctgacgggggttatgcgttcaagtatgaaaacggcaagtacgacattaaagacgtgggcgtggataacgctggcgcgaaagcgggtctgaccttcctggttgacctgattaaaaacaaacacatgaatgcagacaccgattactccatcgcagaagctgcctttaataaaggcgaaacagcgatgaccatcaacggcccgtgggcatggtccaacatcgacaccagcaaagtgaattatggtgtaacggtactgccgaccttcaagggtcaaccatccaaaccgttcgttggcgtgctgagcgcaggtattaacgccgccagtccgaacaaagagctggcaaaagagttcctcgaaaactatctgctgactgatgaaggtctggaagcggttaataaagacaaaccgctgggtgccgtagcgctgaagtcttacgaggaagagttggcgaaagatccacgtattgccgccaccatggaaaacgcccagaaaggtgaaatcatgccgaacatcccgcagatgtccgctttctggtatgccgtgcgtactgcggtgatcaacgccgccagcggtcgtcagactgtcgatgaagccctgaaagacgcgcagactaattcgagctcgaacaacaacaacaataacaataacaacaacctcgggatcgagggaaggatttcagaattcgccctcgcagagggcactcggatcttcgatccggtcaccggtacaacgcatcgcatcgaggatgttgtcggtgggcgcaagcctattcatgtcgtggctgctgccaaggacggaacgctgcatgcgcggcccgtggtgtcctggttcgaccagggaacgcgggatgtgatcgggttgcggatcgccggtggcgccatcctgtgggcgacacccgatcacaaggtgctgacagagtacggctggcgtgccgccggggaactccgcaagggagacagggtggcgcaaccgcgacgcttcgatggattcggtgacagtgcgccgattccggcgcgccgagtgcgggagctgctgctggacgccctgagccccgagcagctagtgctcaccctcctggaggctgagccgccccatgtgctgatcagccgccccagtgcgcccttcaccgaggcctccatgatgatgtccctgaccaagttggccgacaaggagttggtacacatgatcagctgggccaagaagattcccggctttgtggagctcagcctgttcgaccaagtgcggctcttggagagctgttggatggaggtgttaatgatggggctgatgtggcgctcaattgaccaccccggcaagctcatctttgctccagatcttgttctggacagggatgaggggaaatgcgtagaaggaattctggaaatctttgacatgctcctggcaactacttcaaggtttcgagagttaaaactccaacacaaagaatatctctgtgtcaaggccatgatcctgctcaattccagtatgtaccctctggtcacagcgacccaggatgctgacagcagccggaagctggctcacttgctgaacgccgtgaccgatgctttggtttgggtgattgccaagagcggcatctcctcccagcagcaatccatgcgcctggctaacctcctgatgctcctgtcccacgtcaggcatgcgagtaacaagggcatggaacatctgctcaacatgaagtgcaaaaatgtggtcccagtgtatgacctgctgctggagatgctgaatgcccacgtgcttcgcgggtgcaagcgcgtgcaggcgctagcggatgccctggatgacaaattcctgcacgacatgctggcggaagaactccgctattccgtgatccgagaagtgctgccaacgcggcgggcacgaacgttcgacctcgaggtcgaggaactgcacaccctcgtcgccgaaggggttgttgtacacaacatgcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattgggcggccgcactcgagcaccaccaccaccaccactga

SEQ ID NO: 4 - protein, 1075 aa, Artificial
  Amino Acid Sequence Of Estrogen Receptor Beta Biosensor Protein Including Maltose Binding Protein
MKIEEGKLVIWINGDKGYNGLAEVGKKFEKDTGIKVTVEHPDKLEEKFPQVAATGDGPDIIFWAHDRFGGYAQSGLLAEITPDKAFQDKLYPFTWDAVRYNGKLIAYPIAVEALSLIYNKDLLPNPPKTWEEIPALDKELKAKGKSALMFNLQEPYFTWPLIAADGGYAFKYENGKYDIKDVGVDNAGAKAGLTFLVDLIKNKHMNADTDYSIAEAAFNKGETAMTINGPWAWSNIDTSKVNYGVTVLPTFKGQPSKPFVGVLSAGINAASPNKELAKEFLENYLLTDEGLEAVNKDKPLGAVALKSYEEELAKDPRIAATMENAQKGEIMPNIPQMSAFWYAVRTAVINAASGRQTVDEALKDAQTNSSSNNNNNNNNNNLGIEGRISEFALAEGTRIFDPVTGTTHRIEDVVGGRKPIHVVAAAKDGTLHARPVVSWFDQGTRDVIGLRIAGGAILWATPDHKVLTEYGWRAAGELRKGDRVAQPRRFDGFGDSAPIPARRVRELLLDALSPEQLVLTLLEAEPPHVLISRPSAPFTEASMMMSLTKLADKELVHMISWAKKIPGFVELSLFDQVRLLESCWMEVLMMGLMWRSIDHPGKLIFAPDLVLDRDEGKCVEGILEIFDMLLATTSRFRELKLQHKEYLCVKAMILLNSSMYPLVTATQDADSSRKLAHLLNAVTDALVWVIAKSGISSQQQSMRLANLLMLLSHVRHASNKGMEHLLNMKCKNVVPVYDLLLEMLNAHVLRGCKRVQALADALDDKFLHDMLAEELRYSVIREVLPTRRARTFDLEVEELHTLVAEGVVVHNMHPETLVKVKDAEDQLGARVGYIELDLNSGKILESFRPEERFPMMSTFKVLLCGAVLSRIDAGQEQLGRRIHYSQNDLVEYSPVTEKHLTDGMTVRELCSAAITMSDNTAANLLLTTIGGPKELTAFLHNMGDHVTRLDRWEPELNEAIPNDERDTTMPVAMATTLRKLLTGELLTLASRQQLIDWMEADKVAGPLLRSALPAGWFIADKSGAGERGSRGIIAALGPDGKPSRIVVIYTTGSQATMDERNRQIAEIGASLIKHW

SEQ ID NO: 5 - DNA, 2328 bp, Artificial
  Polynucleotide Sequence Encoding Thyroid Receptor Beta Biosensor Protein Including Chitin Binding Domain Protein
atgacgacaaatcctggtgtatccgcttggcaggtcaacacagcttatactgcgggacaattggtcacatataacggcaagacgtataaatgtttgcagccccacacctccttggcaggatgggaaccatccaacgttcctgccttgtggcagcttcaaaacaataacaacaacctcgggatcgagggaaggatttcagaattcgccctcgcagagggcactcggatcttcgatccggtcaccggtacaacgcatcgcatcgaggatgttgtcggtgggcgcaagcctattcatgtcgtggctgctgccaaggacggaacgctgcatgcgcggcccgtggtgtcctggttcgaccagggaacgcgggatgtgatcgggttgcggatcgccggtggcgccatcctgtgggcgacacccgatcacaaggtgctgacagagtacggctggcgtgccgccggggaactccgcaagggagacagggtggcgcaaccgcgacgcttcgatggattcggtgacagtgcgccgattccggcgcgcctgcagaagtccatcgggcacaagccagagcccacagacgaggaatgggagctcatcaaaactgtcaccgaagcccatgtggcgaccaacgcccaaggcagccactggaagcaaaaacggaaattcctgccagaagacattggacaagcaccaatagtcaatgccccagaaggtggaaaggttgacttggaagccttcagccattttacaaaaatcatcacaccagcaattaccagagtggtggattttgccaaaaagttgcctatgttttgtgagctgccatgtgaagaccagatcatcctcctcaaaggctgctgcatggagatcatgtcccttcgcgctgctgtgcgctatgacccagaaagtgagactttaaccttgaatggggaaatggcagtgacacggggccagctgaaaaatgggggtcttggggtggtgtcagacgccatctttgacctgggcatgtctctgtcttctttcaacctggatgacactgaagtagccctccttcaggccgtcctgctgatgtcttcagatcgcccggggcttgcctgtgttgagagaatagaaaagtaccaagatagtttcctgctggcctttgaacactatatcaattaccgaaaacaccacgtgacacacttttggccaaaactcctgatgaaggtgacagatctgcggatgataggagcctgccatgccagccgcttcctgcacatgaaggtggaatgccccacagaactcttcccccctttgttcttggaagtgttcgaggatcgcgtgcaggcgctagcggatgccctggatgacaaattcctgcacgacatgctggcggaagaactccgctattccgtgatccgagaagtgctgccaacgcggcgggcacgaacgttcgacctcgaggtcgaggaactgcacaccctcgtcgccgaaggggttgttgtacacaacggcggcggcggttcttgtatgcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattgggcggccgcactcgagcaccaccaccaccaccac

SEQ ID NO: 6 - protein, 776 aa, Artificial
  Amino Acid Sequence Of Thyroid Receptor Beta Biosensor Protein Including Chitin Binding Domain Protein
MTTNPGVSAWQVNTAYTAGQLVTYNGKTYKCLQPHTSLAGWEPSNVPALWQLQNNNNNLGIEGRISEFALAEGTRIFDPVTGTTHRIEDVVGGRKPIHVVAAAKDGTLHARPVVSWFDQGTRDVIGLRIAGGAILWATPDHKVLTEYGWRAAGELRKGDRVAQPRRFDGFGDSAPIPARLQKSIGHKPEPTDEEWELIKTVTEAHVATNAQGSHWKQKRKFLPEDIGQAPIVNAPEGGKVDLEAFSHFTKIITPAITRVVDFAKKLPMFCELPCEDQIILLKGCCMEIMSLRAAVRYDPESETLTLNGEMAVTRGQLKNGGLGVVSDAIFDLGMSLSSFNLDDTEVALLQAVLLMSSDRPGLACVERIEKYQDSFLLAFEHYINYRKHHVTHFWPKLLMKVTDLRMIGACHASRFLHMKVECPTELFPPLFLEVFEDRVQALADALDDKFLHDMLAEELRYSVIREVLPTRRARTFDLEVEELHTLVAEGVVVHNGGGGSCMHPETLVKVKDAEDQLGARVGYIELDLNSGKILESFRPEERFPMMSTFKVLLCGAVLSRIDAGQEQLGRRIHYSQNDLVEYSPVTEKHLTDGMTVRELCSAAITMSDNTAANLLLTTIGGPKELTAFLHNMGDHVTRLDRWEPELNEAIPNDERDTTMPVAMATTLRKLLTGELLTLASRQQLIDWMEADKVAGPLLRSALPAGWFIADKSGAGERGSRGIIAALGPDGKPSRIVVIYTTGSQATMDERNRQIAEIGASLIKHWAAALEHHHHHH

SEQ ID NO: 7 - DNA, 2307 bp, Artificial
  Polynucleotide Sequence Encoding Estrogen Receptor Beta Biosensor Protein Including Chitin Binding Domain Protein
atgacgacaaatcctggtgtatccgcttggcaggtcaacacagcttatactgcgggacaattggtcacatataacggcaagacgtataaatgtttgcagccccacacctccttggcaggatgggaaccatccaacgttcctgccttgtggcagcttcaaaacaataacaacaacctcgggatcgagggaaggatttcagaattcgccctcgcagagggcactcggatcttcgatccggtcaccggtacaacgcatcgcatcgaggatgttgtcggtgggcgcaagcctattcatgtcgtggctgctgccaaggacggaacgctgcatgcgcggcccgtggtgtcctggttcgaccagggaacgcgggatgtgatcgggttgcggatcgccggtggcgccatcctgtgggcgacacccgatcacaaggtgctgacagagtacggctggcgtgccgccggggaactccgcaagggagacagggtggcgcaaccgcgacgcttcgatggattcggtgacagtgcgccgattccggcgcgccgagtgcgggagctgctgctggacgccctgagccccgagcagctagtgctcaccctcctggaggctgagccgccccatgtgctgatcagccgccccagtgcgcccttcaccgaggcctccatgatgatgtccctgaccaagttggccgacaaggagttggtacacatgatcagctgggccaagaagattcccggctttgtggagctcagcctgttcgaccaagtgcggctcttggagagctgttggatggaggtgttaatgatggggctgatgtggcgctcaattgaccaccccggcaagctcatctttgctccagatcttgttctggacagggatgaggggaaatgcgtagaaggaattctggaaatctttgacatgctcctggcaactacttcaaggtttcgagagttaaaactccaacacaaagaatatctctgtgtcaaggccatgatcctgctcaattccagtatgtaccctctggtcacagcgacccaggatgctgacagcagccggaagctggctcacttgctgaacgccgtgaccgatgctttggtttgggtgattgccaagagcggcatctcctcccagcagcaatccatgcgcctggctaacctcctgatgctcctgtcccacgtcaggcatgcgagtaacaagggcatggaacatctgctcaacatgaagtgcaaaaatgtggtcccagtgtatgacctgctgctggagatgctgaatgcccacgtgcttcgcgggtgcaagcgcgtgcaggcgctagcggatgccctggatgacaaattcctgcacgacatgctggcggaagaactccgctattccgtgatccgagaagtgctgccaacgcggcgggcacgaacgttcgacctcgaggtcgaggaactgcacaccctcgtcgccgaaggggttgttgtacacaacggcggcggcggttcttgtatgcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattgggcggccgcactcgagcaccaccaccaccaccac

SEQ ID NO: 8 - protein, 769 aa, Artificial
  Amino Acid Sequence Of Estrogen Receptor Beta Biosensor Protein Including Chitin Binding Domain Protein
MTTNPGVSAWQVNTAYTAGQLVTYNGKTYKCLQPHTSLAGWEPSNVPALWQLQNNNNNLGIEGRISEFALAEGTRIFDPVTGTTHRIEDVVGGRKPIHVVAAAKDGTLHARPVVSWFDQGTRDVIGLRIAGGAILWATPDHKVLTEYGWRAAGELRKGDRVAQPRRFDGFGDSAPIPARRVRELLLDALSPEQLVLTLLEAEPPHVLISRPSAPFTEASMMMSLTKLADKELVHMISWAKKIPGFVELSLFDQVRLLESCWMEVLMMGLMWRSIDHPGKLIFAPDLVLDRDEGKCVEGILEIFDMLLATTSRFRELKLQHKEYLCVKAMILLNSSMYPLVTATQDADSSRKLAHLLNAVTDALVWVIAKSGISSQQQSMRLANLLMLLSHVRHASNKGMEHLLNMKCKNVVPVYDLLLEMLNAHVLRGCKRVQALADALDDKFLHDMLAEELRYSVIREVLPTRRARTFDLEVEELHTLVAEGVVVHNGGGGSCMHPETLVKVKDAEDQLGARVGYIELDLNSGKILESFRPEERFPMMSTFKVLLCGAVLSRIDAGQEQLGRRIHYSQNDLVEYSPVTEKHLTDGMTVRELCSAAITMSDNTAANLLLTTIGGPKELTAFLHNMGDHVTRLDRWEPELNEAIPNDERDTTMPVAMATTLRKLLTGELLTLASRQQLIDWMEADKVAGPLLRSALPAGWFIADKSGAGERGSRGIIAALGPDGKPSRIVVIYTTGSQATMDERNRQIAEIGASLIKHWAAALEHHHHHH